SEQUENCE LISTING (21 sequences)

SEQ ID NO: 1 - DNA, 1754 bp, Homo sapiens
acagagctccctcccaggcccgcgaacttggccattcagccgccgctgtccccgctgcgcgccctcgcgcctctgcctgagaagccaggcgctgttcccccaccccagaagaggatggcaaaggtggctaaggacctcaacccaggagttaaaaagatgtccctgggccagctgcagtcagcaagaggtgtggcatgtttgggatgcaaggggacgtgttcgggcttcgagccacattcatggaggaaaatatgcaagtcttgcaaatgcagccaagaggaccactgcctaacatctgacctagaagacgatcggaaaattggccgcttgctgatggactccaagtattccaccctcactgctcgggtgaaaggcggggacggcatccggatttacaagaggaaccggatgatcatgaccaaccctattgctactgggaaagatcccacttttgacaccatcacctacgagtgggctccccctggagtcacccagaaactgggactgcagtacatggagctcatccccaaggagaagcagccagtgacaggcacagagggtgccttttaccgccgccgccagctcatgcaccagctccccatctatgaccaggatccctcgcgctgccgtggacttttggagaatgagttgaaactgatggaagaatttgtcaagcaatataagagcgaggccctcggcgtgggagaagtggccctcccggggcagggtggcttgcccaaggaggaggggaagcagcaggaaaagccagagggggcagagaccactgctgctaccaccaacggcagtctcagtgacccgtccaaagaagtggaatacgtctgcgagctctgcaagggagcggcccctcctgacagccccgtggtctactcggacagggcaggctacaacaagcagtggcaccccacctgctttgtgtgtgccaagtgctccgagccgctggtggacctcatctacttctggaaggatggtgcaccctggtgcggccgccattactgcgagagtctgcggccccggtgctccggctgcgatgagataatattcgctgaggactaccagcgtgtggaagatctggcctggcaccgaaagcactttgtctgtgagggttgtgagcagctgctgagcggccgggcgtacatcgtcaccaagggtcagcttctgtgcccaacttgcagcaagtccaaacgctcctgaagggctgcccacccacagccagaatccacaggatcccaccgagaaggagagccaggtgtgccgagaccatcctaagggtccgatgtgacagcaagcaagtgaaataaacaatgatttgcttttcagtgagaatatatatatgagatatatatagatatatattctaggttgggtggtggtagatccttgagggtcagtagtttcaaaaccaaaaatattctaagaagtcttaggatggagttccttttctttctgttgttgtttcccagctacaaccaactaaagacacaaatggcgttctgcaaggggactctgggaggagttttccagaatgcaattccgagtgagcaaatcgcatagctgtagaatgtgcgtgcttttttgtggacacaggagctcctccaggagcaggctgggatcccaactatcgcttgttgcctctttttcaagtggaatttgaattttaaataaacaactttttttggcatgataaacagatcaataaaagttttgtgaattccaaaaaaaaaaaaaaaaaaaaa

SEQ ID NO: 2 - protein, 365 aa, Homo sapiens
MAKVAKDLNPGVKKMSLGQLQSARGVACLGCKGTCSGFEPHSWRKICKSCKCSQEDHCLTSDLEDDRKIGRLLMDSKYSTLTARVKGGDGIRIYKRNRMIMTNPIATGKDPTFDTITYEWAPPGVTQKLGLQYMELIPKEKQPVTGTEGAFYRRRQLMHQLPIYDQDPSRCRGLLENELKLMEEFVKQYKSEALGVGEVALPGQGGLPKEEGKQQEKPEGAETTAATTNGSLSDPSKEVEYVCELCKGAAPPDSPVVYSDRAGYNKQWHPTCFVCAKCSEPLVDLIYFWKDGAPWCGRHYCESLRPRCSGCDEIIFAEDYQRVEDLAWHRKHFVCEGCEQLLSGRAYIVTKGQLLCPTCSKSKRS

SEQ ID NO: 3 - protein, 62 aa, Artificial Sequence
  Description of Artificial Sequence Synthetic zinc-finger motif consensus sequence
CXXCXXXXXXXXXXXXXXXXXXXXXXXHXXXXXCXXCXXXXXXXXXXXXXXXXXXXXXCXXX

SEQ ID NO: 4 - DNA, 21 bp, Artificial Sequence
  Description of Artificial Sequence Synthetic primer
cagtacatgaagctcatcccc

SEQ ID NO: 5 - DNA, 21 bp, Artificial Sequence
  Description of Artificial Sequence Synthetic primer
ggggatgagcttcatgtactg

SEQ ID NO: 6 - DNA, 21 bp, Artificial Sequence
  Description of Artificial Sequence Synthetic primer
gacccgtccagagaagtggaa

SEQ ID NO: 7 - DNA, 21 bp, Artificial Sequence
  Description of Artificial Sequence Synthetic primer
ttccacttctctggacgggtc

SEQ ID NO: 8 - DNA, 59 bp, Artificial Sequence
  Description of Artificial Sequence Synthetic oligonucleotide
ccgggaatgagttgaaactgatggactcgagtccatcagtttcaactcattcttttttg

SEQ ID NO: 9 - DNA, 57 bp, Artificial Sequence
  Description of Artificial Sequence Synthetic oligonucleotide
ccgggctaaggagattggtgctgtactcgagtacagcaccaatctccttagcttttt

SEQ ID NO: 10 - DNA, 14 bp, Homo sapiens
tacatggagctcat

SEQ ID NO: 11 - DNA, 13 bp, Homo sapiens
tacatgaagctca

SEQ ID NO: 12 - DNA, 14 bp, Homo sapiens
  modified_base (7)..(7): a, c, t, g, unknown or other
tacatgnagctcat

SEQ ID NO: 13 - DNA, 14 bp, Homo sapiens
  modified_base (8)..(8): a, c, t, g, unknown or other
atgagctncatgta

SEQ ID NO: 14 - DNA, 13 bp, Homo sapiens
tacatggagctca

SEQ ID NO: 15 - DNA, 13 bp, Homo sapiens
cgtccaaagaagt

SEQ ID NO: 16 - DNA, 13 bp, Homo sapiens
cgtccagagaagt

SEQ ID NO: 17 - DNA, 13 bp, Homo sapiens
ccgtccagagaag

SEQ ID NO: 18 - DNA, 1754 bp, Homo sapiens
acagagctccctcccaggcccgcgaacttggccattcagccgccgctgtccccgctgcgcgccctcgcgcctctgcctgagaagccaggcgctgttcccccaccccagaagaggatggcaaaggtggctaaggacctcaacccaggagttaaaaagatgtccctgggccagctgcagtcagcaagaggtgtggcatgtttgggatgcaaggggacgtgttcgggcttcgagccacattcatggaggaaaatatgcaagtcttgcaaatgcagccaagaggaccactgcctaacatctgacctagaagacgatcggaaaattggccgcttgctgatggactccaagtattccaccctcactgctcgggtgaaaggcggggacggcatccggatttacaagaggaaccggatgatcatgaccaaccctattgctactgggaaagatcccacttttgacaccatcacctacgagtgggctccccctggagtcacccagaaactgggactgcagtacatgaagctcatccccaaggagaagcagccagtgacaggcacagagggtgccttttaccgccgccgccagctcatgcaccagctccccatctatgaccaggatccctcgcgctgccgtggacttttggagaatgagttgaaactgatggaagaatttgtcaagcaatataagagcgaggccctcggcgtgggagaagtggccctcccggggcagggtggcttgcccaaggaggaggggaagcagcaggaaaagccagagggggcagagaccactgctgctaccaccaacggcagtctcagtgacccgtccaaagaagtggaatacgtctgcgagctctgcaagggagcggcccctcctgacagccccgtggtctactcggacagggcaggctacaacaagcagtggcaccccacctgctttgtgtgtgccaagtgctccgagccgctggtggacctcatctacttctggaaggatggtgcaccctggtgcggccgccattactgcgagagtctgcggccccggtgctccggctgcgatgagataatattcgctgaggactaccagcgtgtggaagatctggcctggcaccgaaagcactttgtctgtgagggttgtgagcagctgctgagcggccgggcgtacatcgtcaccaagggtcagcttctgtgcccaacttgcagcaagtccaaacgctcctgaagggctgcccacccacagccagaatccacaggatcccaccgagaaggagagccaggtgtgccgagaccatcctaagggtccgatgtgacagcaagcaagtgaaataaacaatgatttgcttttcagtgagaatatatatatgagatatatatagatatatattctaggttgggtggtggtagatccttgagggtcagtagtttcaaaaccaaaaatattctaagaagtcttaggatggagttccttttctttctgttgttgtttcccagctacaaccaactaaagacacaaatggcgttctgcaaggggactctgggaggagttttccagaatgcaattccgagtgagcaaatcgcatagctgtagaatgtgcgtgcttttttgtggacacaggagctcctccaggagcaggctgggatcccaactatcgcttgttgcctctttttcaagtggaatttgaattttaaataaacaactttttttggcatgataaacagatcaataaaagttttgtgaattccaaaaaaaaaaaaaaaaaaaaa

SEQ ID NO: 19 - DNA, 1754 bp, Homo sapiens
acagagctccctcccaggcccgcgaacttggccattcagccgccgctgtccccgctgcgcgccctcgcgcctctgcctgagaagccaggcgctgttcccccaccccagaagaggatggcaaaggtggctaaggacctcaacccaggagttaaaaagatgtccctgggccagctgcagtcagcaagaggtgtggcatgtttgggatgcaaggggacgtgttcgggcttcgagccacattcatggaggaaaatatgcaagtcttgcaaatgcagccaagaggaccactgcctaacatctgacctagaagacgatcggaaaattggccgcttgctgatggactccaagtattccaccctcactgctcgggtgaaaggcggggacggcatccggatttacaagaggaaccggatgatcatgaccaaccctattgctactgggaaagatcccacttttgacaccatcacctacgagtgggctccccctggagtcacccagaaactgggactgcagtacatggagctcatccccaaggagaagcagccagtgacaggcacagagggtgccttttaccgccgccgccagctcatgcaccagctccccatctatgaccaggatccctcgcgctgccgtggacttttggagaatgagttgaaactgatggaagaatttgtcaagcaatataagagcgaggccctcggcgtgggagaagtggccctcccggggcagggtggcttgcccaaggaggaggggaagcagcaggaaaagccagagggggcagagaccactgctgctaccaccaacggcagtctcagtgacccgtccagagaagtggaatacgtctgcgagctctgcaagggagcggcccctcctgacagccccgtggtctactcggacagggcaggctacaacaagcagtggcaccccacctgctttgtgtgtgccaagtgctccgagccgctggtggacctcatctacttctggaaggatggtgcaccctggtgcggccgccattactgcgagagtctgcggccccggtgctccggctgcgatgagataatattcgctgaggactaccagcgtgtggaagatctggcctggcaccgaaagcactttgtctgtgagggttgtgagcagctgctgagcggccgggcgtacatcgtcaccaagggtcagcttctgtgcccaacttgcagcaagtccaaacgctcctgaagggctgcccacccacagccagaatccacaggatcccaccgagaaggagagccaggtgtgccgagaccatcctaagggtccgatgtgacagcaagcaagtgaaataaacaatgatttgcttttcagtgagaatatatatatgagatatatatagatatatattctaggttgggtggtggtagatccttgagggtcagtagtttcaaaaccaaaaatattctaagaagtcttaggatggagttccttttctttctgttgttgtttcccagctacaaccaactaaagacacaaatggcgttctgcaaggggactctgggaggagttttccagaatgcaattccgagtgagcaaatcgcatagctgtagaatgtgcgtgcttttttgtggacacaggagctcctccaggagcaggctgggatcccaactatcgcttgttgcctctttttcaagtggaatttgaattttaaataaacaactttttttggcatgataaacagatcaataaaagttttgtgaattccaaaaaaaaaaaaaaaaaaaaa

SEQ ID NO: 20 - protein, 365 aa, Homo sapiens
MAKVAKDLNPGVKKMSLGQLQSARGVACLGCKGTCSGFEPHSWRKICKSCKCSQEDHCLTSDLEDDRKIGRLLMDSKYSTLTARVKGGDGIRIYKRNRMIMTNPIATGKDPTFDTITYEWAPPGVTQKLGLQYMKLIPKEKQPVTGTEGAFYRRRQLMHQLPIYDQDPSRCRGLLENELKLMEEFVKQYKSEALGVGEVALPGQGGLPKEEGKQQEKPEGAETTAATTNGSLSDPSKEVEYVCELCKGAAPPDSPVVYSDRAGYNKQWHPTCFVCAKCSEPLVDLIYFWKDGAPWCGRHYCESLRPRCSGCDEIIFAEDYQRVEDLAWHRKHFVCEGCEQLLSGRAYIVTKGQLLCPTCSKSKRS

SEQ ID NO: 21 - protein, 365 aa, Homo sapiens
MAKVAKDLNPGVKKMSLGQLQSARGVACLGCKGTCSGFEPHSWRKICKSCKCSQEDHCLTSDLEDDRKIGRLLMDSKYSTLTARVKGGDGIRIYKRNRMIMTNPIATGKDPTFDTITYEWAPPGVTQKLGLQYMELIPKEKQPVTGTEGAFYRRRQLMHQLPIYDQDPSRCRGLLENELKLMEEFVKQYKSEALGVGEVALPGQGGLPKEEGKQQEKPEGAETTAATTNGSLSDPSREVEYVCELCKGAAPPDSPVVYSDRAGYNKQWHPTCFVCAKCSEPLVDLIYFWKDGAPWCGRHYCESLRPRCSGCDEIIFAEDYQRVEDLAWHRKHFVCEGCEQLLSGRAYIVTKGQLLCPTCSKSKRS

(19) United States
(12) Patent Application Publication
(10) Pub. No.: US 2013/0239239 A1
(43) Pub. Date: Sep. 12, 2013

(54) LMCD1 CANCER MARKERS AND METHODS FOR THEIR USE

(71) Applicants: Y-S Jou, Taipei (TW); C-Y Chang, Taipei (TW)

(72) Inventors: Y-S Jou, Taipei (TW); C-Y Chang, Taipei (TW)

(73) Assignee: Academia Sinica

(21) Appl. No.: 13/414,679

(22) Filed: Mar. 7, 2012

(51) Int. Cl. G01N 33/574; C12Q 1/02; C12N 15/85; C12N 5/10; A61K 39/395; C07H 21/04; C07K 16/18; A01K 67/027; A61P 35/00; C12Q 1/68; A61K 31/7088

(52) U.S. Cl. 800/10; 436/503; 435/6.11; 435/7.1; 435/29; 435/320.1; 435/325; 514/44 A; 536/24.31; 530/389.7; 424/174.1

(57) ABSTRACT

The present invention provides LMCD1 cancer markers, and methods, compositions, and kits for their use. The invention also provides expression vectors, host cells, and transgenic animals comprising one or more LMCD1 mutations, and methods for their use in characterizing, diagnosing, and treating cancers, and for identifying potential therapeutics. The invention also provides cancer therapeutics.

### BACKGROUND OF THE INVENTION

LMCD1 (LIM and cysteine-rich domains 1, dyxin) is a member of LIM protein family containing two C-terminal LIM domains, a central PET domain and a N-terminal cysteine-rich region. LIM domains contain two contiguous zinc-finger motifs with a defined consensus sequence CX₂CX₁₆₋₂₃HX₂(C/H)X₂CX₂CX₁₆₋₂₁CX₂(C/H/D), and such domains are mostly observed in proteins associated with actin cytoskeleton to regulate cell adhesion or migration. LMCD1 has been previously described as a transcriptional corepressor of GATA6.

Cancer is a group of more than 100 diseases in which a group of cells display uncontrolled growth (cell division beyond the normal limits). In most cases, cancer cells form a clump of cells called a tumor, although in some cancers, like leukemia, the cells do not form tumors. Tumors may be malignant or benign. Cancers typically comprise cells with abnormal genetic material and usually undergo rapid uncontrolled cell growth, invade and destroy adjacent tissue, and sometimes spread to other locations in the body, such as via lymph or blood. Twenty percent of Americans die from cancer, half due to lung, breast, and colon-rectal cancer, and skin cancer remains a serious health hazard. In the year 2000, there were 553,091 cancer deaths in the US. One-third of all individuals in the United States will develop cancer during their life. Although the five-year survival rate has risen dramatically as a result of progress in early diagnosis and therapy, cancer still remains second only to cardiac disease as a cause of death in the United States.

Liver cancer is the sixth most common cancer in the world and the third most common cause of cancer mortality. Hepatocellular carcinoma (HCC) is the most common type of liver cancer. Tumorigenesis of HCC is a slowly progressive and multistep process with several known etiological factors including hepatitis viral infection (hepatitis B or C virus), consumption of aflatoxin B1 contaminated foods, and alcohol abuse. Repeated exposure to these etiological agents may induce inflammatory cell injury and consequent rounds of necrosis and proliferation. Liver cirrhosis is commonly observed after these deteriorated hepatocytes are replaced with regenerative nodules surrounded by collagen fibrous scarring. The hyperplastic nodules may further evolve into pre-malignant dysplastic nodules and eventually progress to HCC accompanied with increasing genomic instability and accumulated chromosomal alterations.

Recurrent chromosomal alterations in cancers are commonly associated with putative cancer genes. In HCC, many tumor suppressor genes and oncogenes have been identified based on genetic lesions, for example: loss of TP53 (17p13), RB and BRCA2 (13q), and amplification of c-myc (8q24) and ERBB2 (17q12-q21). Epigenetic mechanisms can also contribute to HCC, such as CpG hypermethylation of p16(INK4a) and COX2, as well as altered expression of miR-122 and miR-21. Point mutation is another common mechanism to alter functions of cancer genes. In HCC, frequent point mutations of p53 and β-catenin are involved in key pathways of hepatocarcinogenesis. Other studies have reported HCC mutations in Axin1, Ras, M6P/IGF2R, Smad2/4 and PTEN. However, HCC remains a highly lethal cancer due to the lack of biomarkers and targets for early diagnosis, categorization and therapeutic interventions. Metastasis also remains a serious concern for cancers arising from many other tissues.

Thus, there is a need for additional biomarkers for the identification, characterization, and/or treatment of cancers, particularly with regard to liver cancer.

### SUMMARY OF THE INVENTION

In one aspect, the invention provides a method for characterizing a cancer tissue. In one embodiment, the method comprises: (a) providing a sample of a subject; (b) assaying said sample for the presence or absence of one or more LMCD1 mutations by detecting the presence or absence of the one or more LMCD1 mutations or a SNP in high linkage disequilibrium with said one or more LMCD1 mutations, wherein the presence of said one or more LMCD1 mutations indicates a higher propensity for metastasis than is indicated by the absence of said one or more LMCD1 mutations; and (c) reporting the result of step (b) to a designated person or entity. In some embodiments, the one or more LMCD1 mutations is one or more of G517A and A824G. In some embodiments, the one or more LMCD1 mutations is one or more of E135K and K237R. In some embodiments, the cancer tissue comprises hepatocellular carcinoma. In some embodiments, assaying comprises contacting a nucleic acid derived from the sample with an oligonucleotide in a hybridization reaction. In some embodiments, the oligonucleotide is a probe that specifically hybridizes to a target sequence comprising an LMCD1 mutation, wherein detection of hybridization between the probe and the target sequence indicates the presence of the LMCD1 mutation. In some embodiments, the oligonucleotide is a primer and the hybridization reaction comprises a sequencing reaction, whereby a target sequence comprising an LMCD1 mutation is detected by extension of the primer. In some embodiments, assaying comprises contacting a protein derived from the sample with a binding element specific for an LMCD1 mutant protein, wherein detection of binding between the binding element and the LMCD1 mutant protein indicates the presence of one of said one or more LMCD1 mutations. In some embodiments, the binding element is an antibody. In some embodiments, the method further comprises administering a therapeutic agent based on the results of step (b). In some embodiments, the therapeutic agent is an inhibitor that downregulates Rac1 output. In some embodiments, downregulation of Rac1 output is indicated by one or more of: (a) a decreased number of migrated cells in a trans-well migration assay; (b) a decrease in migration velocity of cells in a wound-healing assay; (c) a decrease in migration velocity of cells in a random migration assay; (d) a decrease in the percentage of cells with lamellipodia; and (e) a decrease in Rac1 expression at the nucleic acid or protein level; wherein (a)-(e) compare treated and untreated cells expressing LMCD1 comprising said one or more LMCD1 mutations. In some embodiments, a higher propensity for metastasis induced by the one or more LMCD1 mutations is indicated by one or more of: (a) an increased number of migrated cells in a trans-well migration assay; (b) an increase in migration velocity of cells in a wound-healing assay; (c) an increase in migration velocity of cells in a random migration assay; and (d) an increase in the percentage of cells with lamellipodia; wherein (a)-(d) compare cells expressing LMCD1 comprising said one or more LMCD1 mutations and cells expressing LMCD1 lacking said one or more LMCD1 mutations.

In one aspect, the invention provides a method of predicting propensity of cancer cells to metastasize. In one embodiment, the method comprises: (a) providing a sample of a subject; (b) assaying said sample for the presence or absence of one or more LMCD1 mutations; and (c) predicting the propensity of said cancer cells to metastasize based on the presence or absence of the one or more LMCD1 mutations. In some embodiments, the presence of said one or more LMCD1 mutations indicates a higher propensity for metastasis than is indicated by the absence of said one or more LMCD1 mutations. In some embodiments, the one or more LMCD1 mutations is one or more of G517A and A824G. In some embodiments, the one or more LMCD1 mutations is one or more of E135K and K237R. In some embodiments, the cancer tissue comprises hepatocellular carcinoma. In some embodiments, assaying comprises contacting a nucleic acid derived from the sample with an oligonucleotide in a hybridization reaction. In some embodiments, the oligonucleotide is a probe that specifically hybridizes to a target sequence comprising an LMCD1 mutation, wherein detection of hybridization between the probe and the target sequence indicates the presence of the LMCD1 mutation. In some embodiments, the oligonucleotide is a primer and the hybridization reaction comprises a sequencing reaction, whereby a target sequence comprising an LMCD1 mutation is detected by extension of the primer. In some embodiments, assaying comprises contacting a protein derived from the sample with a binding element specific for an LMCD1 mutant protein, wherein detection of binding between the binding element and the LMCD1 mutant protein indicates the presence of one of said one or more LMCD1 mutations. In some embodiments, the binding element is an antibody. In some embodiments, the method further comprises administering a therapeutic agent based on the results of step (b). In some embodiments, the therapeutic agent is an inhibitor that downregulates Rac1 output. In some embodiments, downregulation of Rac1 output is indicated by one or more of: (a) a decreased number of migrated cells in a trans-well migration assay; (b) a decrease in migration velocity of cells in a wound-healing assay; (c) a decrease in migration velocity of cells in a random migration assay; (d) a decrease in the percentage of cells with lamellipodia; and (e) a decrease in Rac1 expression at the nucleic acid or protein level; wherein (a)-(e) compare treated and untreated cells expressing LMCD1 comprising said one or more LMCD1 mutations. In some embodiments, a higher propensity for metastasis induced by the one or more LMCD1 mutations is indicated by one or more of: (a) an increased number of migrated cells in a trans-well migration assay; (b) an increase in migration velocity of cells in a wound-healing assay; (c) an increase in migration velocity of cells in a random migration assay; and (d) an increase in the percentage of cells with lamellipodia; wherein (a)-(d) compare cells expressing LMCD1 comprising said one or more LMCD1 mutations and cells expressing LMCD1 lacking said one or more LMCD1 mutations.

In one aspect, the invention provides a method of treating cancer in a subject. In one embodiments, the method comprises: (a) providing a sample of a subject; (b) assaying said sample for the presence or absence of one or more LMCD1 mutations, wherein the presence of said one or more LMCD1 mutations indicates a higher propensity for metastasis than is indicated by the absence of said one or more LMCD1 mutations; and (c) treating said subject with an inhibitor that reduces metastasis, if said one or more LMCD1 mutations is present. In some embodiments, the inhibitor downregulates Rac1 output. In some embodiments, the inhibitor comprises a nucleic acid trigger that triggers the RNA-interference pathway to decrease expression of LMCD1 or Rac1, such as an shRNA, an miRNA, an siRNA, an antisense RNA, and/or a double-stranded RNA. In some embodiments, the inhibitor is selected from the group consisting of polypeptides, antibodies, and small molecules. In some embodiments, the inhibitor is selected from the group consisting of W56, F56, EHT 1864, Rac1 Inhibitor II, NSC23760, and NSC23766. In some embodiments, the one or more LMCD1 mutations is one or more of G517A and A824G. In some embodiments, the one or more LMCD1 mutations is one or more of E135K and K237R. In some embodiments, the cancer tissue comprises hepatocellular carcinoma. In some embodiments, assaying comprises contacting a nucleic acid derived from the sample with an oligonucleotide in a hybridization reaction. In some embodiments, the oligonucleotide is a probe that specifically hybridizes to a target sequence comprising an LMCD1 mutation, wherein detection of hybridization between the probe and the target sequence indicates the presence of the LMCD1 mutation. In some embodiments, the oligonucleotide is a primer and the hybridization reaction comprises a sequencing reaction, whereby a target sequence comprising an LMCD1 mutation is detected by extension of the primer. In some embodiments, assaying comprises contacting a protein derived from the sample with a binding element specific for an LMCD1 mutant protein, wherein detection of binding between the binding element and the LMCD1 mutant protein indicates the presence of one of said one or more LMCD1 mutations. In some embodiments, the binding element is an antibody. In some embodiments, reduction in metastasis is indicated by one or more of: (a) a decreased number of migrated cells in a trans-well migration assay; (b) a decrease in migration velocity of cells in a wound-healing assay; (c) a decrease in migration velocity of cells in a random migration assay; (d) a decrease in the percentage of cells with lamellipodia; and (e) a decrease in Rac1 expression at the nucleic acid or protein level; wherein (a)-(e) compare treated and untreated cells expressing LMCD1 comprising said one or more LMCD1 mutations.

In one aspect, the invention provides a method for screening inhibitors of cancer metastasis. In one embodiments, the method comprises: (a) providing a cell line expressing LMCD1 having one or more LMCD1 mutations; (b) exposing said cell line to a test compound; and (c) determining the effect of said compound on cell migration, wherein a decrease in cell migration of cells treated with said compound compared to cells not treated with said compound identifies said compound as an inhibitor of cancer metastasis. In some embodiments, the test compound comprises a nucleic acid trigger that triggers the RNA-interference pathway, such as an shRNA, an miRNA, an siRNA, an antisense RNA, and/or a double-stranded RNA. In some embodiments, the test compound is selected from the group consisting of polypeptides, nucleic acids, small organic molecules, small inorganic molecules, ligands, aptamers, antibodies, radiation, and light of one or more selected frequencies. In some embodiments, the one or more LMCD1 mutations is one or more of G517A and A824G. In some embodiments, the one or more LMCD1 mutations is one or more of E135K and K237R. In some embodiments, a decrease in cell migration is indicated by one or more of: (a) a decreased number of migrated cells in a trans-well migration assay; (b) a decrease in migration velocity of cells in a wound-healing assay; (c) a decrease in migration velocity of cells in a random migration assay; and (d) a decrease in the percentage of cells with lamellipodia; wherein (a)-(d) compare cells expressing LMCD1 comprising said one or more LMCD1 mutations and cells expressing LMCD1 lacking said one or more LMCD1 mutations. In some embodiments, cells of the cell line have a genome comprising an integrated (e.g. stably integrated) transgenic nucleotide sequence encoding a mutant LMCD1. In some embodiments, (i) cells of the cell line are transplanted into a tissue of a non-human animal before step (b); (ii) cells of the cell line are exposed to the test compound by administering the test compound to the non-human animal; and (iii) the decrease in cell migration is indicated by a decrease in the number of the cells found in a tissue that is not the tissue into which the cells were transplanted. In some embodiments, (i) cells of the cell line are transplanted into a tissue of a non-human animal after step (b); and (ii) the decrease in cell migration is indicated by a decrease in the number of the cells found in a tissue that is not the tissue into which the cells were transplanted.

In one aspect, the invention provides an isolated oligonucleotide for the detection of an LMCD1 mutation. In one embodiment, the oligonucleotide comprises at least 6 nucleotides complementary to a target sequence comprising said LMCD1 mutation, wherein said at least 6 nucleotides comprise at least one nucleotide complementary to said LMCD1 mutation, and further wherein said isolated oligonucleotide is substantially complementary to said target sequence. In some embodiments, the LMCD1 mutation is G517A or A824G. In some embodiments, the isolated oligonucleotide further comprises a detectable label. In some embodiments, the isolated oligonucleotide further comprises a quencher, such that a signal from the detectable label is only detectable when the quencher is removed from the oligonucleotide. In some embodiments, the isolated oligonucleotide is linked to a solid substrate.

In one aspect, the invention provides an isolated antibody, or antigen-binding antibody fragment thereof directed specifically to a human mutant protein, or protein fragment thereof comprising one or more LMCD1 mutations. In some embodiments, the one or more LMCD1 mutations is one or more of E135K and K237R. In some embodiments, the isolated antibody binds an epitope predicted to be on the surface of the LMCD1 mutant protein. In some embodiments, the isolated antibody is a monoclonal antibody, and/or a humanized antibody. In some embodiments, the isolated antibody binds an epitope within a PET domain of the LMCD1 mutant protein, or within a LIM domain of the LMCD1 mutant protein. In some embodiments, the isolated antibody comprises a detectable label.

In one aspect, the invention provides an expression vector comprising a nucleotide sequence encoding a mutant LMCD1 comprising one or more LMCD1 mutations, wherein expression of said mutant LMCD1 increases cell mobility in cells that actively express said expression vector. In some embodiments, the one or more LMCD1 mutations is one or more of G517A and A824G. In some embodiments, the one or more LMCD1 mutations is one or more of E135K and K237R. In some embodiments, the expression vector further comprises a selectable marker. In some embodiments, the expression vector further encodes a detectable label. In some embodiments, the increase in cell mobility is indicated by one or more of: (a) an increased number of migrated cells in a trans-well migration assay; (b) an increase in migration velocity of cells in a wound-healing assay; (c) an increase in migration velocity of cells in a random migration assay; and (d) an increase in the percentage of cells with lamellipodia; wherein (a)-(d) compare cells expressing said expression vector and cells not expressing said expression vector.

In one aspect, the invention provides a transgenic cell whose genome comprises an integrated (e.g. stably integrated) transgenic nucleotide sequence encoding a mutant LMCD1 comprising one or more LMCD1 mutations. In one embodiment, the transgenic cell actively expresses said mutant LMCD1 and exhibits increased cell mobility relative to a cell not expressing said mutant LMCD1. In some embodiments, the one or more LMCD1 mutations is one or more of G517A and A824G. In some embodiments, the one or more LMCD1 mutations is one or more of E135K and K237R. In some embodiments, the transgenic cell expresses a selectable marker, and/or a detectable label. In some embodiments, the increased mobility is indicated by one or more of: (a) an increased number of migrated cells in a trans-well migration assay; (b) an increase in migration velocity of cells in a wound-healing assay; (c) an increase in migration velocity of cells in a random migration assay; and (d) an increase in the percentage of cells with lamellipodia; wherein (a)-(d) compare cells expressing said mutant LMCD1 and cells not expressing said mutant LMCD1.

In one aspect, the invention provides a non-human transgenic animal whose genome comprises an integrated (e.g. stably integrated) nucleotide sequence encoding a mutant LMCD1 comprising one or more LMCD1 mutations. In one embodiment, cancer cells arising from the cells of said non-human transgenic animal and expressing said mutant LMCD1 exhibit a higher propensity for metastasis relative to cancer cells not expressing said mutant LMCD1. In some embodiments, the one or more LMCD1 mutations is one or more of G517A and A824G. In some embodiments, the one or more LMCD1 mutations is one or more of E135K and K237R. In some embodiments, cells of the non-human transgenic animal express a selectable marker, and/or a detectable label. In some embodiments, a higher propensity for metastasis induced by said one or more LMCD1 mutations is indicated by one or more of: (a) an increased number of migrated cells in a trans-well migration assay; (b) an increase in migration velocity of cells in a wound-healing assay; (c) an increase in migration velocity of cells in a random migration assay; and (d) an increase in the percentage of cells with lamellipodia; wherein (a)-(d) compare cells expressing LMCD1 comprising said one or more LMCD1 mutations and cells expressing LMCD1 lacking said one or more LMCD1 mutations. In some embodiments, the higher propensity for metastasis induced by expression of said mutant LMCD1 is indicated by an increase in the number of metastatic growths in a tissue from which the cells of the metastatic growth did not originate.

In one aspect, the invention provides a kit for assaying for a mutation of LMCD1. In one embodiment, the kit comprises (a) one or more reagents suitable for detecting a mutant LMCD1 comprising one or more LMCD1 mutations; and (b) instructions for using said one or more reagents. In some embodiments, the one or more LMCD1 mutations is one or more of G517A and A824G. In some embodiments, the one or more LMCD1 mutations is one or more of E135K and K237R.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1a illustrates results of a DNA copy number alteration study in multiple human cancer cell lines, indicating the location of a commonly amplified region.

FIG. 1b illustrates relative upregulation of LMCD1 in HCC tissues.

FIG. 1c shows the results of semi-quantitative PCR of LMCD1 in HCC cell lines and tumor pairs.

FIG. 1d shows a schematic of LMCD1, the relative location of domains and mutations, and the identity of LMCD1 mutations identified in HCC tumor tissues.

FIG. 2a shows a Western blot analysis of LMCD1 transfectants in SK-Hep1 cells.

FIG. 2b shows the percentage of cells with lamellipodia formation within a group of 100 selected cells.

FIG. 2c shows the results of immunofluorescent staining of mock and transfected cells with anti-Arp3 antibody.

FIG. 2d shows double staining of cells with LMCD1 (anti-Flag antibody) and F-actin (rhodamine-phalloidin).

FIG. 3a shows the results of a proliferation assay of mock and LMCD1 transfectants in SK-hep1 cells.

FIG. 3b shows representative images of anchorage-independent growth assays of LMCD1 transfectants.

FIG. 3c shows the results of a wound-healing assay, with photographs taken at zero and 20 hours after wounding (bar=500 μm)

FIG. 3d shows plotted tracks of representative migrating cells during wound healing, and quantification of migration velocity.

FIG. 3e shows representative images of a trans-well migration assay, an quantification of migrated cell number.

FIG. 4a shows a Western blot analysis of LMCD1 knockdown efficiency in multiple stable clones infected with lentiviral shLMCD1 or luciferase shRNA (shCtrl) as control.

FIG. 4b shows images of cells treated with various inhibitors decreasing lamellipodia formation (bar=50 μm).

FIG. 4c shows the results of shRNA treatment on wound healing in a wound healing assays.

FIG. 4d shows the results of a trans-well migration assay in PLC/PRF/5 cells treated with estradiol, and optionally an shRNA.

FIG. 4e shows a quantification of results from FIG. 4d, and results of reverse transcription-PCR with LMCD1-specific primer pairs.

FIG. 5a shows a Western blot analysis of Rac1 expression in LMCD1 transfectants.

FIGS. 5b-e show the effects of Rac1 inhibitors (NSC23766 and Rac1 shRNA) on Rac1 expression and wound closure.

FIG. 6 shows an increase in tumor metastasis to lung in a mouse model induced by the E135K mutation in LMCD1.

FIG. 7 shows images of cells at the edge of a wound, with cells expressing mutant LMCD1 having enhanced lemellipodia protrusion (bar=50 μm).

FIG. 8 shows plotted tracks of representative migrating cells in a random migration assay, and quantification of migration velocity.

FIG. 9 shows images from wound healing and trans-well migration assays in SK-Hep1 cells stably transfected with LMCD1 mutants and control cells (bar=500 μm).

FIG. 10 shows the results of trans-well migration assay in stable LMCD1 transfectants in Huh7 cells, quantification of migrated cell number, and Western blot for LMCD1 protein expression.

FIG. 11 shows the results of LMCD1 knockdown by shRNA on trans-well migration and wound-healing assays (bar=500 μm), as well as the effect on LMCD1 expression determined by semi-quantitative PCR.

FIG. 12 shows LMCD1 overexpression in 377 of 949 HCC patient tissues.

FIG. 13 shows LMCD1 mutations detected in tumor tissue and adjacent to normal tissue lacking the mutations.

### DEFINITIONS

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two sequences that hybridize under stringent conditions.

As used herein, “stringent conditions” for hybridization refer to conditions under which a nucleic acid having complementarity to a target sequence predominantly hybridizes with a target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y. Conditions that increase the stringency of a hybridization reaction are widely known and published in the art. See, for example, (Sambrook, et al., (1989), supra; Nonradioactive In Situ Hybridization Application Manual, Boehringer Mannheim, second edition). Examples of relevant conditions include (in order of increasing stringency): incubation temperatures of 25° C., 37° C., 50° C., and 68° C.; buffer concentrations of 10×SSC, 6×SSC, 1×SSC, 0.1×SSC (where SSC is 0.15 M NaCl and 15 mM citrate buffer) and their equivalent using other buffer systems; formamide concentrations of 0%, 25%, 50%, and 75%, incubation times from 5 minutes to 24 hours and washes of increasing duration, increasing frequency, or decreasing buffer concentrations. In a non-limiting example of a low-stringency hybridization reaction, hybridization is carried out at about 40° C. in 10×SSC or a solution of equivalent ionic strength/temperature. In a non-limiting example of a moderate-stringency hybridization, hybridization is performed at about 50° C. in 6×SSC. In a non-limiting example of a high-stringency hybridization reaction, hybridization is performed at about 60° C. in 1×SSC.

The terms “polypeptide”, “peptide” and “protein” are used interchangeably herein to refer to polymers of amino acids of any length. The polymer may be linear or branched, it may comprise modified amino acids, and it may be interrupted by non amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to effect beneficial or desired results. The therapeutically effective amount will vary depending upon the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose will vary depending on the particular agent chosen, the dosing regimen to be followed, whether it is administered in combination with other compounds, timing of administration, the tissue to be imaged, and the physical delivery system in which it is carried.

### DETAILED DESCRIPTION OF THE INVENTION

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

In general, the invention relates to LMCD1 mutations. An LMCD1 gene or LMCD1 protein comprising one or more changes to a wild-type or normal sequence is referred to as an “LMCD1 mutant,” and each change is referred to as an “LMCD1 mutation.” Mutations include any change to the reference sequence, including but not limited to deletion, insertion, substitution, and combinations of these. Changes at the nucleic acid level may be coding or non-coding, and includes deletion of one or more nucleotides, insertion of one or more nucleotides, and/or substitution of one or more nucleotides for one or more different nucleotides. With respect to changes in the coding sequence of the LMCD1 gene, SEQ ID NO:1 provides the reference wild-type mRNA sequence, changes to which constitute LMCD1 mutations. Non-limiting examples of LMCD1 mutations include G517A and A824G. G517A refers to the change of the guanine at position 517 of SEQ ID NO:1 to an adenine. A824G refers to the change of an adenine at position 824 of SEQ ID NO:1 to a guanine. In some embodiments, copy number (e.g. genotype) of an LMCD1 mutation is determined. With respect to changes in the amino acid sequence of LMCD1 protein, SEQ ID NO:2 provides the reference wild-type amino acid sequence, changes to which constitutes LMCD1 mutations contained in LMCD1 mutant proteins. Non-limiting examples of LMCD1 mutations include E135K, K237R, and R154C. E135K refers to the change of the glutamic acid at position 135 of SEQ ID NO:2 to a lysine. K237R refers to the change of the lysine at position 237 of SEQ ID NO:2 to an arginine. R154C refers to the change of the arginine at position 154 of SEQ ID NO:2 to a cysteine. In some embodiments, one or more LMCD1 mutations are associated with and/or induce increased cell mobility and/or increase in propensity for cancer metastasis. As such, LMCD1 mutations are useful for cancer detection, characterization, prognosis, prediction of metastasis, and/or treatment of cancer.

In some embodiments, a single nucleotide polymorphism (SNP) linked to an LMCD1 mutation may be used as a marker for the presence or absence of the LMCD1 mutation to which it is linked, such that the LMCD1 mutation is detected indirectly through detection of the linked SNP. Thus, assaying for an LMCD1 mutation can comprise direct and/or indirect detection. In some embodiments, a linked SNP used as a marker for the presence or absence of the LMCD1 mutation to which it is linked is in linkage disequilibrium (LD) with the linked LMCD1 mutation, such as high linkage disequilibrium. Methods for measuring LD are known in the art. One common measure of LD is the coefficient of linkage disequilibrium (D), which represents the difference between the observed frequency of a haplotype and the frequency expected on the basis of a random association of alleles in gametes. In general, D=P_{AB}*P_{ab}−P_{aB}*P_{Ab}, where P_{xy} is the frequency of the haplotype xy. D ranges from −0.25 to 0.25, with high absolute values of D corresponding to high linkage disequilibrium between two linked loci. In some embodiments, high linkage disequilibrium corresponds to an absolute value of the coefficient of linkage disequilibrium of about or more than about 0.05, 0.1, 0.15, 0.16, 0.17, 0.18, 0.19, 0.2, 0.21, 0.22, 0.23, 0.24, or higher. In some embodiments, high linkage disequilibrium comprises a deviation from random association by about or more than about 10%, 20%, 30%, 40%, 50%, 60%, 70%, 80%, 90%, 95%, or higher. Another common measure of LD is r². In some embodiments, high linkage disequilibrium comprises an r² value about or higher than about 0.7, 0.8, 0.85, 0.9, 0.95, 0.97, 0.98, 0.99, or higher. Methods for measuring linkage disequilibrium are discussed in US 20060177863, which is incorporated herein by reference in its entirety. In some embodiments, a selected linked SNP is used as an alternative maker in specific populations (e.g. geographic populations and ethnic groups) where the linked SNP and the LMCD1 mutation are in high LD.

In one aspect, the invention provides a method of characterizing a cancer tissue. In one embodiment, the method comprises (a) providing a sample of a subject; (b) assaying said sample for the presence or absence of one or more LMCD1 mutations, wherein the presence of said one or more LMCD1 mutations indicates a higher propensity for metastasis than is indicated by the absence of said one or more LMCD1 mutations; and (c) reporting the results of step (b) to a designated person or entity. In another aspect, the invention provides a method of predicting propensity of cancer cells to metastasize. In one embodiment, the method comprises (a) providing a sample of a subject; (b) assaying said sample for the presence or absence of one or more LMCD1 mutations; and (c) predicting the propensity of said cancer cells to metastasize based on the presence or absence of the one or more LMCD1 mutations.

Typically, a sample of a subject comprises cancerous or pre-cancerous cells. Samples may be obtained by any suitable means, including but not limited to needle aspiration, fine needle aspiration, core needle biopsy, vacuum assisted biopsy, large core biopsy, incisional biopsy, excisional biopsy, punch biopsy, shave biopsy, skin biopsy, and venipuncture. A sample may be analyzed directly for its contents, or may be processed to purify one or more of its contents for analysis. Methods of direct analysis of samples are known in the art and include, without limitation, mass spectrometry and histological staining procedures. In some embodiments, one or more components are purified from the sample for the detection of an LMCD1 mutation. In some embodiments, the purified component of the sample is a nucleic acid, such as DNA (e.g. genomic DNA) or RNA (e.g. total RNA or mRNA). In some embodiments, the purified component of the sample is protein (e.g. total protein, cytoplasmic protein, or membrane protein). Methods for the purification of nucleic acids and/or proteins from a sample are known in the art.

Any cancer may be analyzed according to the methods of the invention. Many kinds of cancers are known in the art. Examples of types of cancers include, without limitation, cancers originating from epithelial cell tissue (carcinomas), blood cells (leukemias, lymphomas, myelomas), connective tissue (sarcomas), or glial or supportive cells (gliomas). In some embodiments, the target cancers are carcinomas and/or blood cell malignancies. In some embodiments, the target cancers are lung tumors, breast tumors, ovarian tumors, pancreatic tumors, glioblastoma tumors, and/or sarcomas. Cancers may comprise solid and/or non-solid tumors. Cancers may comprise primary and/or secondary tumors. Non-limiting examples of cancers that may be analyzed according to the methods of the invention include Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Müllerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, primary or secondary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Szary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, and Wilms' tumor. Other non-limiting examples of cancer types are described in U.S. Pat. No. 7,405,227, incorporated herein by reference in its entirety.

In some embodiments, a sample is assayed to determine whether or not cells of a cancer have a higher propensity for metastasis. As used herein, “metastasis” refers to the spread of cancer from one organ, tissue, or location in the subject to another. A cancer treatment that has efficacy in “inhibiting” metastasis refers to a treatment that reduces the spread of cancer cells to areas in the subject beyond the initial tumor relative to the spread of cancer cells in a similar subject in the absence of the treatment. Conversely, a cancer treatment is ineffective in inhibiting metastasis if that treatment results in the movement of cancer cells that is not significantly less than the movement of cancer cells in a similar subject in the absence of the treatment. A propensity for metastasis may be measured in terms of capacity for or likelihood of a cancer or its constituent cells to metastasize, and may be expressed in relation to other cells of the cancer, cells of other cancers, non-cancerous cells of the same or different tissue type, or with respect to a unit of time (e.g. number of metastasizing cells per unit time or average time per metastatic event; also referred to as the rate of metastasis). For comparison between different cells or tissues, a higher propensity for metastasis may be measured by an increase in the number of metastasized cells under specified conditions for cells of one type (e.g. a cancer under analysis) as compared to cells of another type (e.g. a reference). In some embodiments, a higher propensity for metastasis is indicated by a capacity for increased mobility, such as may be conferred by the presence of one or more mutations with a demonstrated enhancing effect on mobility, such as one or more LMCD1 mutations. A number of assays for determining the effect of a mutation on cell mobility are known in the art, and include without limitation, trans-well migration assays, wound healing assays, random migration assays, and morphological analyses of lamellipodia formation. In some embodiments, a higher propensity for metastasis indicates a likelihood of metastasis of a cell or tissue having one or more LMCD1 mutations that is about or more than about 5%, 10%, 15%, 20%, 25%, 30%, 35%, 40%, 45%, 50%, 55%, 60%, 70%, 80%, 90%, 100%, or higher than the likelihood of metastasis of a reference cell or tissue lacking the one or more LMCD1 mutations. In some embodiments, a higher propensity for metastasis of a cell or tissue having one or more LMCD1 mutations indicates a likelihood of metastasis that is about or more than about 2-fold, 3-fold, 4-fold, 5-fold, 6-fold, 7-fold, 8-fold, 9-fold, 10-fold, 20-fold, 50-fold, 100-fold, 1000-fold, or more than a reference cell or tissue lacking the one or more LMCD1 mutations. The effects of a mutation on a cell's propensity for metastasis may differ depending on the genotype of the mutation. For example, a cell that is homozygous for one or more LMCD1 mutations may have the same or higher propensity for metastasis than a cell that is heterozygous for the one or more LMCD1 mutations, which in turn have a higher propensity for metastasis than a cell having no copies of the one or more LMCD1 mutation (i.e. homozygous wild-type at the one or more corresponding nucleotide or amino acid positions). Thus, in some embodiments, the method further comprises determining the genotype of one or more LMCD1 mutations.

In some embodiments, the association of one or more LMCD1 mutations with a higher propensity for metastasis is used to predict the propensity of cancer cells to metastasize. In general, a prediction refers to a forecast of the likelihood that a cancer will metastasize, such as within a given period of time. A prediction of likelihood may be with respect to a reference cell or tissue (e.g. cancer cells of the same type having the same one or more LMCD1 mutations, no LMCD1 mutations, or an uncharacterized LMCD1 gene). In some embodiments, the prediction indicates that metastasis is about, less than about, or more than about 5%, 10%, 15%, 20%, 25%, 30%, 35%, 40%, 45%, 50%, 55%, 60%, 70%, 80%, 90%, 100%, or more likely compared to a reference cell or tissue. In some embodiments, the prediction indicates that metastasis is about, less than about, or more than about 2-fold, 3-fold, 4-fold, 5-fold, 6-fold, 7-fold, 8-fold, 9-fold, 10-fold, 20-fold, 50-fold, 100-fold, 1000-fold, or more likely compared to a reference cell or tissue.

In some embodiments, a higher propensity for metastasis associated with and/or induced by one or more LMCD1 mutations is determined by a trans-well migration assay. Methods for performing a trans-well assay are known in the art, and the conditions of such an assay may be optimized for a particular cell type, environment, or other relevant factor known to those skilled in the art. In one example trans-well assay, cells are seeded into the upper chamber of a porous insert. The insert is placed into a chamber with 10% FBS as a chemoattractant, and allowed to migrate towards the attractant for a period of time, such as 24 hours. Unmigrated cells on the seeded side of the membrane are removed. Cells that migrated across the membrane through the pores remain and are counted, such as through the use of a stain. An increase in the number of migrated cells in a trans-well assay indicates that one or more LMCD1 mutations expressed in said cells increase cell migration, which in turn is associated with a higher propensity for metastasis. In some embodiments, the number of migrated cells expressing a mutant LMCD1 having one or more LMCD1 mutations increases by about, or more than about 5%, 10%, 15%, 20%, 25%, 30%, 35%, 40%, 45%, 50%, 55%, 60%, 70%, 80%, 90%, 100%, or more compared to cells not expressing the mutant LMCD1. In some embodiments, the number of migrated cells expressing a mutant LMCD1 having one or more LMCD1 mutations increases by about or more than about 2-fold, 3-fold, 4-fold, 5-fold, 6-fold, 7-fold, 8-fold, 9-fold, 10-fold, 20-fold, 50-fold, 100-fold, 1000-fold, or more compared to cells not expressing the mutant LMCD1.

In some embodiments, a higher propensity for metastasis associated with and/or induced by one or more LMCD1 mutations is determined by a wound-healing assay. Methods for performing a wound-healing assay are known in the art, and the conditions of such an assay may be optimized for a particular cell type, environment, or other relevant factor known to those skilled in the art. In one example wound-healing assay, cells are seeded on a plate at a confluent density and cultured to grow into a monolayer. The culture is scratched, such as with a pipette tip, and allowed to close for a period of time, such as 20 hours. The average reduction of distance between the two wound edges is divided by the period of time allowed for closing to calculate a migration velocity. An increase in migration velocity in a wound-healing assay indicates that one or more LMCD1 mutations expressed in said cells increase cell migration, which in turn is associated with a higher propensity for metastasis. In some embodiments, the migration velocity of cells expressing a mutant LMCD1 having one or more LMCD1 mutations increases by about, or more than about 5%, 10%, 15%, 20%, 25%, 30%, 35%, 40%, 45%, 50%, 55%, 60%, 70%, 80%, 90%, 100%, or more compared to cells not expressing the mutant LMCD1. In some embodiments, the migration velocity of cells expressing a mutant LMCD1 having one or more LMCD1 mutations increases by about, or more than about 2-fold, 3-fold, 4-fold, 5-fold, 6-fold, 7-fold, 8-fold, 9-fold, 10-fold, 20-fold, 50-fold, 100-fold, 1000-fold, or more compared to cells not expressing the mutant LMCD1.

In some embodiments, a higher propensity for metastasis associated with and/or induced by one or more LMCD1 mutations is determined by a random migration assay. Methods for performing a wound-healing assay are known in the art, and the conditions of such an assay may be optimized for a particular cell type, environment, or other relevant factor known to those skilled in the art. In one example random-migration assay, cells are sparsely plated in suitable growth medium. Cells are then placed in an environmentally controlled chamber on a microscope equipped with an imaging device, such as a camera. Time-lapse photos of the cells are taken at regular intervals or a period of time sufficient to track movement, such as every 15 minutes for 18 hours. Tracks of one or more individual cells are plotted, and distance travelled is divided by time elapsed to arrive at a migration velocity, which may be averaged across multiple cells of the analyzed type, such as on the same plate. Velocities of individual cells may be similarly determined for cells in other assays, such as a wound healing assay. An increase in the migration velocity of cells in a random migration assay indicates that one or more LMCD1 mutations expressed in said cells increase cell migration, which in turn is associated with a higher propensity for metastasis. In some embodiments, the migration velocity of cells expressing a mutant LMCD1 having one or more LMCD1 mutations increases by about, or more than about 5%, 10%, 15%, 20%, 25%, 30%, 35%, 40%, 45%, 50%, 55%, 60%, 70%, 80%, 90%, 100%, or more compared to cells not expressing the mutant LMCD1. In some embodiments, the migration velocity of cells expressing a mutant LMCD1 having one or more LMCD1 mutations increases by about or more than about 2-fold, 3-fold, 4-fold, 5-fold, 6-fold, 7-fold, 8-fold, 9-fold, 10-fold, 20-fold, 50-fold, 100-fold, 1000-fold, or more compared to cells not expressing the mutant LMCD1.

In some embodiments, a higher propensity for metastasis associated with and/or induced by one or more LMCD1 mutations is determined by morphological analysis of cells for the presence of, and optionally the number of, lamellipodia. Morphological analysis generally includes the use of a microscope to view cells from a sample of a subject or cells gown in culture. Morphological analysis may include the use of stains, many of which are known in the art. Stains may be specific or non-specific. In some embodiments, morphological analysis includes the use of a stain specific for one or more marker of lamellipodia, such as a stain for Arp3. An increase in the percentage of cells with lamellipodia indicates that one or more LMCD1 mutations expressed in the cells increases cell migration, which in turn is associated with a higher propensity for metastasis. In some embodiments, the percentage of cells with lamellipodia in a sample of cells expressing a mutant LMCD1 having one or more LMCD1 mutations increases by about, or more than about 5%, 10%, 15%, 20%, 25%, 30%, 35%, 40%, 45%, 50%, 55%, 60%, 70%, 80%, 90%, 100%, or more compared to cells not expressing the mutant LMCD1. In some embodiments, the percentage of cells with lamellipodia in a sample of cells expressing a mutant LMCD1 having one or more LMCD1 mutation increases by about or more than about 2-fold, 3-fold, 4-fold, 5-fold, 6-fold, 7-fold, 8-fold, 9-fold, 10-fold, 20-fold, 50-fold, 100-fold, 1000-fold, or more compared to cells not expressing the mutant LMCD1.

Hybridization

In some embodiments, detection of an LMCD1 mutation comprises detection of an LMCD1 nucleic acid in or from a sample of a subject, such as DNA and/or RNA. Detection of nucleic acids typically involves the use of a hybridization reaction, such as between a target nucleic acid and an oligonucleotide probe or primer. Accordingly, in one aspect, the invention provides an isolated oligonucleotide for the detection of one or more LMCD1 mutations. An oligonucleotide used in a nucleic acid detection reaction may be of any length sufficient to achieve a desired level of specificity under the conditions of a given assay. An oligonucleotide may be about, less than about, or more than about 5, 6, 7, 8, 9, 10, 11, 12, 13, 14, 15, 16, 17, 18, 19, 20, 25, 30, 35, 36, 40, 45, 50, 60, 70, 80, 90, 100, or more nucleotides in length. In some embodiments, the isolated oligonucleotide comprises at least 6 nucleotides complementary to a target sequence comprising an LMCD1 mutation, wherein said at least 6 nucleotides comprise at least one nucleotide complementary to said LMCD1 mutation, and further wherein said isolated oligonucleotide is substantially complementary to said target sequence. In some embodiments, the oligonucleotide comprises one or more detectable labels. In some embodiments, an oligonucleotide comprises a FRET pair of labels, such as may be used in real-time PCR amplification reactions.

In some embodiments, the oligonucleotide is immobilized on a substrate. Substrates include, but are not limited to, arrays, microarrays, wells of a multi-well plate, and beads (e.g. non-magnetic, magnetic, paramagnetic, hydrophobic, and hydrophilic beads). Examples of materials useful as substrates include but are not limited to nitrocellulose, glass, silicon, and a variety of gene arrays. A preferred hybridization assay is conducted on high-density gene chips as described in U.S. Pat. No. 5,445,934.

In general, “hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self hybridizing strand, or any combination of these. A hybridization reaction may constitute a step in a more extensive process, such as the initiation of an amplification reaction (PCR, ligase chain reaction, or a sequencing reaction), or the enzymatic cleavage of a polynucleotide by an endonuclease. A sequence capable of hybridizing with a given sequence is referred to as the “complement” of the given sequence. A target nucleic acid to which an oligonucleotide binds will comprise a target sequence, which is the one or more nucleotides of the target nucleic acids whose identity is sought to be determined. In some embodiments, an oligonucleotide hybridizes directly to the target sequence, or one or more nucleotides thereof, such as in the case of a probe, the binding of which to the target sequence is indicative of the presence of the target sequence. In some embodiments, the target sequence comprises an LMCD1 mutation, and detection of the specific hybridization of an oligonucleotide probe to the target sequence is indicative of the presence of the LMCD1 mutation. In some embodiments, two oligonucleotides hybridize to a target nucleic acid, adjacent to one another, and in close proximity to the target sequence (e.g. within about 1, 2, 3, 4, 5, 6, 7, 8, 9, 10, 11, 12, 13, 14, 15, 16, 17, 18, 19, or 20 bases), or with one of the two oligonucleotide probes directly hybridizing to one or more nucleotides of the target sequence, for example when detection comprises the formation of a ligation product comprising the two oligonucleotides. In some embodiments, the oligonucleotide hybridizes adjacent to the target sequence, such that detection of the target sequence is achieved by extension of the oligonucleotide by a polymerase, such as in a sequencing or real-time PCR reaction. Hybridization adjacent to a target sequence may be within any distance of the target sequence sufficient to achieve detection, such as within about 1, 2, 3, 4, 5, 6, 7, 8, 9, 10, 11, 12, 13, 14, 15, 16, 17, 18, 19, 20, 25, 50, 100, 150, 200, 300, 400, 500, 600, 700, 800, 900, 1000, or more nucleotides of the target sequence. In some embodiments, the target sequence is a mutation comprising one or more nucleotide changes with respect to a reference sequence (e.g. a wild-type sequence). In some embodiments, the mutation consists of a single nucleotide, and constitutes a single-nucleotide polymorphism (SNP).

An oligonucleotide useful in detections methods, such as probes and primers, can be a nucleic acid comprising the nucleotide sequence of a coding strand or its complementary strand, such as of a target DNA, or the nucleotide sequence of a sense strand or antisense strand, such as of a target RNA. An oligonucleotide can comprise DNA and/or RNA and can bind DNA and/or RNA in the biological sample. An oligonucleotide may alternatively, or additionally, comprise modified nucleotides (e.g. methylated or labeled nucleotides), modified backbone chemistries (e.g. morpholine ring-containing backbones), nucleotide analogues, and combinations of these. An oligonucleotide can be the coding or complementary strand of a complete gene or gene fragment. The nucleotide sequence of an oligonucleotide can be any sequence having sufficient complementarity to a nucleic acid sequence in the biological sample to allow for hybridization of an oligonucleotide to the target nucleic acid in the biological sample under a desired hybridization condition. Preferably, an oligonucleotide will hybridize only to the nucleic acid target of interest in the sample and will not bind non-specifically to other non-complementary nucleic acids in the sample or other regions of the target nucleic acid in the sample. The hybridization conditions can be varied according to the degree of stringency desired in the in situ hybridization. For example, if the hybridization conditions are for high stringency, an oligonucleotide will bind only to the nucleic acid sequences in the sample with which it has a very high degree of complementarity. Low stringency hybridization conditions will allow for hybridization of an oligonucleotide to nucleic acid sequences in the sample which have some complementarity but which are not as highly complementary to the oligonucleotide sequence as would be required for hybridization to occur at high stringency. The hybridization conditions will vary depending on the biological sample, oligonucleotide type and target. An artisan will know how to optimize hybridization conditions for a particular application of a selected method.

An oligonucleotide can be commercially obtained or can be synthesized according to standard nucleotide synthesizing protocols well known in the art. Alternatively, an oligonucleotide can be produced by isolation and purification of a nucleic acid sequence from biological materials according to methods standard in the art of molecular biology (Sambrook et al. 1989. Molecular Cloning: A Laboratory Manual, 2d Ed., Cold Spring Harbor Laboratory Pres, Cold Spring Harbor, N.Y.).

It is further contemplated that the present invention also includes methods for oligonucleotide hybridization wherein the hybridized oligonucleotide is used as a primer for an enzyme catalyzed elongation reaction such as in situ PCR and primed in situ labeling reactions whereby haptenized nucleotides are incorporated in situ. Additionally included are methods for in situ hybridization, employing synthetic peptide nucleic acid (PNA) oligonucleotide probes (Nielsen et al., 1991. “Sequence-selective recognition of DNA by strand displacement with a thymine-substituted polyamide.” Science 254:1497-1500; Egholm et al., 1993. “PNA hybridizes to complementary oligonucleotides obeying the Watson-Crick hydrogen bonding rules.” Nature 365:566-568).

Oligonucleotides may be linked to one or more labels for use as a probe for a target sequence. Non-limiting examples of labels to which an oligonucleotide may linked include a hapten, biotin, digoxigenin, fluorescein isothiocyanate (FITC), dinitrophenyl, amino methyl coumarin acetic acid, acetylaminofluorene and mercury-sulfhydryl-ligand complexes, as well as any other label described herein or known in the art.

Detection of the presence, and optionally amount, of an LMCD1 nucleic acid can be conducted in real time in an amplification assay. In some embodiments, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. Non-limiting examples of DNA-binding dyes suitable for this application include SYBR green, SYBR blue, DAPI, propidium iodine, Hoechst stain, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like. In some embodiments, one or more LMCD1 mutant nucleic acids are amplified while LMCD1 nucleic acids not containing the one or more target mutations are not amplified, such as by use of one or more primers specific for the one or more mutations. In other embodiments, an LMCD1 nucleic acid is amplified without regard to the presence of a specific mutated target sequence, which target sequence is instead detected during or after the amplification reaction, such as by binding of an oligonucleotide probe to the template nucleic acid or amplification product.

In some embodiments, fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan° probes) resulting in increased specificity and sensitivity. Methods for performing probe-based quantitative amplification are well established in the art and are taught in U.S. Pat. No. 5,210,015.

In some embodiments, conventional hybridization assays using hybridization probes complementary with a target LMCD1 mutant sequence can be performed. Typically, probes are allowed to form stable complexes with the target polynucleotides contained within the biological sample derived from a test subject (or nucleic acids derived therefrom) in a hybridization reaction. It will be appreciated by one of skill in the art that where antisense is used as the probe nucleic acid, the target polynucleotides provided in the sample are chosen to be complementary to sequences of the antisense nucleic acids. Conversely, where the nucleotide probe is a sense nucleic acid, the target polynucleotide is selected to be complementary to sequences of the sense nucleic acid.

For convenient detection of the probe-target complexes formed during the hybridization assay, the nucleotide probes can be conjugated to a detectable label. Detectable labels suitable for use in the present invention include any composition detectable by photochemical, biochemical, spectroscopic, immunochemical, electrical, optical or chemical means. A wide variety of appropriate detectable labels are known in the art, which include fluorescent or chemiluminescent labels, radioactive isotope labels, enzymatic or other ligands. In preferred embodiments, one will likely desire to employ a fluorescent label or an enzyme tag, such as digoxigenin, β-galactosidase, urease, alkaline phosphatase or peroxidase, avidin/biotin complex.

The detection methods used to detect or quantify the hybridization intensity will typically depend upon the label selected above. For example, radiolabels may be detected using photographic film or a phosphoimager. Fluorescent markers may be detected and quantified using a photodetector to detect emitted light. Enzymatic labels are typically detected by providing the enzyme with a substrate and measuring the reaction product produced by the action of the enzyme on the substrate; and finally colorimetric labels are detected by simply visualizing the colored label.

In some embodiments, hybridization of a probe to a target sequence (e.g., a SNP or mutation) is detected directly by visualizing a bound probe (e.g., a Northern or Southern assay; See e.g., Ausabel et al. (eds.), Current Protocols in Molecular Biology, John Wiley & Sons, NY (1991)). In a typical blotting assay, genomic DNA (Southern) or RNA (Northern) is isolated from a sample of a subject. The DNA or RNA is then cleaved with a series of restriction enzymes that cleave infrequently in the genome. The DNA or RNA is then separated (e.g., on an agarose gel) and transferred to a membrane. A labeled (e.g., by incorporating a radionucleotide) probe or probes specific for the SNP or mutation being detected is allowed to contact the membrane under a condition of low, medium, or high stringency conditions. Unbound probe is removed and the presence of binding is detected by visualizing the labeled probe.

In some embodiments, variant sequences are detected using an array-based hybridization assay. In this assay, a series of oligonucleotide probes are affixed to a solid support. The oligonucleotide probes are designed to be unique to a given SNP or mutation. The DNA sample of interest is contacted with the oligonucleotide probe array and hybridization is detected.

In some embodiments, the probe array assay is a GeneChip (Affymetrix, Santa Clara, Calif.; See e.g., U.S. Pat. Nos. 6,045,996; 5,925,525; and 5,858,659; each of which is herein incorporated by reference) assay. The GeneChip technology uses miniaturized, high-density arrays of oligonucleotide probes affixed to an array or “chip.” Example probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

In some embodiments, the nucleic acid to be analyzed on a probe array is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In some embodiments, a probe array containing electronically captured probes (Nanogen, San Diego, Calif.) is utilized (See e.g., U.S. Pat. Nos. 6,017,696; 6,068,818; and 6,051,380; each of which are herein incorporated by reference). Through the use of microelectronics, Nanogen's technology enables the active movement and concentration of charged molecules to and from designated test sites on its semiconductor microchip. Nucleic acid capture probes unique to a given SNP or mutation are electronically placed at, or “addressed” to, specific sites on the microchip. Since DNA has a strong negative charge, it can be electronically moved to an area of positive charge. First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample can then be analyzed for the presence of target DNA molecules by determining which of the DNA capture probes hybridize, with complementary DNA in the test sample (e.g., a PCR amplified gene of interest). An electronic charge can also be used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding,

In some embodiments, an array technology based upon the segregation of fluids on a flat surface (chip) by differences in surface tension (ProtoGene, Palo Alto, Calif.) is utilized (See e.g., U.S. Pat. Nos. 6,001,311; 5,985,551; and 5,474,796; each of which is herein incorporated by reference). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered only to the sites where amidite A is to be coupled during that synthesis step and so on. Common reagents and washes are delivered by flooding the entire surface and then removing them by spinning DNA probes unique for the SNP or mutation of interest can be affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In some embodiments, a “bead array” is used for the detection of polymorphisms (Illumina, San Diego, Calif.; See e.g., PCT Publications WO 99/67641 and WO 00/39587, each of which is herein incorporated by reference). Illumina uses a bead array technology that combines fiber optic bundles and beads that self-assemble into an array. Each fiber optic bundle contains thousands to millions of individual fibers depending on the diameter of the bundle. The beads are coated with an oligonucleotide specific for the detection of a given SNP or mutation. Batches of beads are combined to form a pool specific to the array. To perform an assay, the bead array is contacted with a prepared subject sample (e.g., DNA). Hybridization is detected using any suitable method.

In some embodiments, hybridization of a bound probe is detected using a TaqMan assay (PE Biosystems, Foster City, Calif.; See e.g., U.S. Pat. Nos. 5,962,233 and 5,538,848, each of which is herein incorporated by reference). The assay is performed during a PCR reaction. The TaqMan assay exploits the 5′-3′ exonuclease activity of DNA polymerases such as AMPLITAQ DNA polymerase. A probe specific for a given allele (such as an allele of a SNP) or mutation is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ polymerase cleaves the probe between the reporter and the quencher dye. The separation of the reporter dye from the quencher dye results in an increase of fluorescence. The signal accumulates with each cycle of PCR and can be monitored with a fluorimeter.

In some embodiments, polymorphisms are detected using the SNP-IT primer extension assay (Orchid Biosciences, Princeton, N.J.; See e.g., U.S. Pat. Nos. 5,952,174 and 5,919,626, each of which is herein incorporated by reference). In this assay, SNPs are identified by using a specially synthesized DNA primer and a DNA polymerase to selectively extend the DNA chain by one base at the suspected SNP location. DNA in the region of interest is amplified and denatured. Polymerase reactions are then performed using miniaturized systems called microfluidics. Detection is accomplished by adding a label to the nucleotide suspected of being at the SNP or mutation location. Incorporation of the label into the DNA can be detected by any suitable method (e.g., if the nucleotide contains a biotin label, detection is via a fluorescently labeled antibody specific for biotin). Numerous other assays are known in the art.

Additional assays that are suitable for use in methods of detection in the present invention include, but are not limited to, enzyme mismatch cleavage methods (e.g., Variagenics, U.S. Pat. Nos. 6,110,684, 5,958,692, 5,851,770, herein incorporated by reference in their entireties); polymerase chain reaction; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884 and 6,183,960, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); INVADER assay, Third Wave Technologies; See e.g., U.S. Pat. Nos. 5,846,717, 6,090,543; 6,001,567; 5,985,557; and 5,994,069; each of which is herein incorporated by reference; cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (Bamay Proc. Natl. Acad. Sci. USA 88, 189-93 (1991)); and sandwich hybridization methods (e.g., U.S. Pat. No. 5,288,609, herein incorporated by reference in its entirety).

In some embodiments, a nucleic acid hybridization reaction used to identify one or more LMCD1 mutations is a sequencing reaction. In some embodiments, a sequencing reaction comprising extension of an oligonucleotide primer is used to determine the nucleotide sequence of a target sequence comprising an LMCD1 mutation. The template in a sequencing reaction may be nucleic acids isolated from a sample, nucleic acid amplification products, or a combination of these. Sequence analysis using template dependent synthesis can include a number of different processes. For example, in four-color Sanger sequencing methods, a population of template molecules is used to create a population of complementary fragment sequences. Primer extension is carried out in the presence of the four naturally occurring nucleotides, and with a sub-population of dye labeled terminator nucleotides, e.g., dideoxyribonucleotides, where each type of terminator (ddATP, ddGTP, ddTTP, ddCTP) includes a different detectable label. As a result, a nested set of fragments is created where the fragments terminate at each nucleotide in the sequence beyond the primer, and are labeled in a manner that permits identification of the terminating nucleotide. The nested fragment population is then subjected to size based separation, e.g., using capillary electrophoresis, and the labels associated with each different sized fragment is identified to identify the terminating nucleotide. As a result, the sequence of labels moving past a detector in the separation system provides a direct readout of the sequence information of the synthesized fragments, and by complementarity, the underlying template (See, e.g., U.S. Pat. No. 5,171,534, incorporated herein by reference in its entirety for all purposes).

Other examples of template dependent sequencing methods include sequence by synthesis processes, where individual nucleotides are identified iteratively, as they are added to the growing primer extension product. These processes generally fall into two categories. In a first category, a nucleic acid synthesis complex is contacted with one or more nucleotides under conditions that permit the addition of a single base, and little or no extension beyond that base. The reaction is then interrogated or observed to determine whether a base was incorporated, and provide the identity of that base. The second category generally provides for the real-time observation of the addition of nucleotides to the growing nascent strand in an uninterrupted reaction process, e.g., without wash steps.

One example of the first category of processes is pyrosequencing, which is a sequence-by-synthesis process that identifies the incorporation of a nucleotide by assaying the resulting synthesis mixture for the presence of by-products of the sequencing reaction, namely pyrophosphate. In particular, a primer, polymerase template complex is contacted with a single type of nucleotide. If that nucleotide is incorporated, the polymerization reaction cleaves the nucleoside triphosphate between the α and β phosphates of the triphosphate chain, releasing pyrophosphate. The presence of released pyrophosphate is then identified using a chemiluminescent enzyme reporter system that converts the pyrophosphate, with AMP, into ATP, then measures ATP using a luciferase enzyme to produce measurable light signals. Where light is detected, the base is incorporated, where no light is detected, the base is not incorporated. Following appropriate washing steps, the various bases are cyclically contacted with the complex to sequentially identify subsequent bases in the template nucleic acid. See, e.g., U.S. Pat. No. 6,210,891, incorporated herein by reference in its entirety for all purposes).

In certain other related processes, the primer-template-polymerase complex is immobilized upon a substrate and the complex is contacted with labeled nucleotides. In some embodiments, the nucleotides are provided with and without removable terminator groups, and upon incorporation, the label is coupled with the complex and is thus detectable. In the case of terminator-bearing nucleotides, all four different nucleotides, bearing individually identifiable labels, are contacted with the complex. Incorporation of the labeled nucleotide arrests extension, by virtue of the presence of the terminator, and adds the label to the complex. The label and terminator are then removed from the incorporated nucleotide, and following appropriate washing steps, the process is repeated. In the case of non-terminated nucleotides, a single type of labeled nucleotide is added to the complex to determine whether it will be incorporated, as with pyrosequencing. Following removal of the label group on the nucleotide and appropriate washing steps, the various different nucleotides are cycled through the reaction mixture in the same process. See, e.g., U.S. Pat. No. 6,833,246, incorporated herein by reference in its entirety for all purposes). These template-directed sequencing methods that comprise one-at-a-time nucleotide incorporations, e.g., separated by buffer exchange or wash steps, are sometimes referred to as “flush-and-scan” methods, and are typically considered to be non-processive sequence-by-synthesis technologies.

As noted above, in the second category of sequence-by-synthesis processes, the incorporation of differently labeled nucleotides is observed in real time as template-dependent synthesis is carried out in a processive manner. In particular, an individual immobilized polymerase-template-primer complex is observed as fluorescently labeled nucleotides are incorporated, permitting real time identification of each added base as it is added. In this process, label groups are attached to a portion of the nucleotide that is cleaved during incorporation. For example, by attaching the label group to a portion of the phosphate chain removed during incorporation, i.e., a β, γ, or other terminal phosphate group on a nucleoside polyphosphate, the label is not incorporated into the nascent strand, and instead, natural DNA is produced. In preferred aspects, observation of individual molecules typically involves the optical confinement of the complex within a very small illumination volume. By optically confining the complex, one creates a monitored region in which randomly diffusing nucleotides are present for a very short period of time, while incorporated nucleotides are retained within the observation volume for longer as they are being incorporated. This strategy results in a characteristic signal associated with the incorporation event, which is also characterized by a signal profile that is specific for the base being added. In related aspects, interacting label components, such as fluorescent resonant energy transfer (FRET) dye pairs, are provided upon the polymerase or other portion of the complex and the incorporating nucleotide, such that the incorporation event puts the labeling components in interactive proximity, and a characteristic signal results that is, again, also specific for the base being incorporated (See, e.g., U.S. Pat. Nos. 7,056,661, 6,917,726, 7,033,764, 7,052,847, 7,056,676, 7,170,050, 7,361,466, 7,416,844 and Published U.S. Patent Application Nos. 2007-0134128 and 2009-0024331, the full disclosures of which are hereby incorporated herein by reference in their entirety for all purposes).

Mass Spectroscopy Assay

In some embodiments, a MassARRAY system (Sequenom, San Diego, Calif.) is used to detect LMCD1 mutation sequences (see e.g., U.S. Pat. Nos. 6,043,031; 5,777,324; and 5,605,798; each of which is herein incorporated by reference). In a typical MassARRAY assay, DNA is isolated from a sample of a subject (e.g. a blood sample) using standard procedures. Next, specific DNA regions containing the mutation or SNP of interest, about 200 base pairs in length, are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization-Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than one thousandth of a second, enabling samples to be analyzed in a total of 3-5 seconds including repetitive data collection. The SpectroTYPER software then calculates, records, compares and reports the genotypes at the rate of about three seconds per sample.

Protein Assays

In some embodiments, an LMCD1 mutant protein in a sample is identified using a binding element specific for the LMCD1 mutant protein, such that detection of the binding between the LMCD1 mutant protein and the binding element indicates the presence of one or more corresponding LMCD1 mutations. As used herein, a “binding element” refers to any molecule that is capable of specifically binding a target LMCD1 mutant protein. Examples of binding elements include, but are not limited to, polypeptides, nucleic acids, small organic molecules, small inorganic molecules, ligands, aptamers, and antibodies.

In some embodiments, antibodies (or fragments or equivalents thereof) are used in the detection of one or more LMCD1 mutant, or in the treatment of a subjection having a cancer comprising an LMCD1 mutation. Antibody-based detection methods are known in the art, and include without limitation, ELISA, immunohistochemistry, agglutination, Western blotting, and others. Methods of treatment employing antibodies as therapeutic agents are also known in the art, with specific treatment regimens selected based on a variety of parameters, including but not limited to physical characteristics of the subject (e.g. height, weight, sex, and age), type and stage of disease, and whether or not treatment includes additional therapeutic agents.

Methods of producing antibodies are known in the art. Preferred antibodies are isolated, in the sense of being purified to reduce the presence of contaminants such as antibodies able to bind other polypeptides and/or other serum components. Monoclonal antibodies are preferred for some purposes, though polyclonal antibodies are within the scope of the present invention.

Where the kits comprise more than one antibody, these are preferably mixtures of isolated antibodies as described above.

Antibodies may be obtained using techniques which are standard in the art. Methods of producing antibodies include immunizing a mammal (e.g. mouse, rat, rabbit) with a polypeptide comprising the target antigen. Antibodies may be obtained from immunized animals using any of a variety of techniques known in the art, and screened, preferably using binding of antibody to antigen of interest. For instance, Western blotting techniques or immunoprecipitation may be used (Armitage et al, Nature, 357:80-82, 1992).

As an alternative or supplement to immunizing a mammal with a peptide, an antibody specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047.

Antibodies according to the present invention may be modified in a number of ways. The term “antibody” includes any antibody, antigen binding antibody fragments, derivatives, functional equivalents and homologues of antibodies, including synthetic molecules and molecules whose shape mimics that of an antibody enabling it to bind an antigen or epitope. Antibodies also include any protein consisting of one or more polypeptides substantially encoded by all or part of the recognized immunoglobulin genes. The recognized immunoglobulin genes, for example in humans, include the kappa (k), lambda (l), and heavy chain genetic loci, which together comprise the myriad variable region genes, and the constant region genes mu (u), delta (d), gamma (g), sigma (e), and alpha (α) which encode the IgM, IgD, IgG, IgE, and IgA isotypes respectively. Antibody herein is meant to include full length antibodies and antibody fragments, and may refer to a natural antibody from any organism, an engineered antibody, or an antibody generated recombinantly for experimental, therapeutic, or other purposes. The term “antibody” includes antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies. Particularly preferred are full length antibodies that comprise Fc variants. The term “antibody” also includes monoclonal and polyclonal antibodies. Antibodies can be antagonists, agonists, neutralizing, inhibitory, or stimulatory.

The antibodies of the present invention may be nonhuman, chimeric, humanized, or fully human. For a description of the concepts of chimeric and humanized antibodies see Clark et al., 2000 and references cited therein (Clark, 2000, Immunol Today 21:397-402). Chimeric antibodies comprise the variable region of a nonhuman antibody, for example VH and VL domains of mouse or rat origin, operably linked to the constant region of a human antibody (see for example U.S. Pat. No. 4,816,567). In a preferred embodiment, the antibodies of the present invention are humanized. By “humanized” antibody as used herein is meant an antibody comprising a human framework region (FR) and one or more complementarity determining regions (CDR's) from a non-human (such as mouse or rat) antibody. The non-human antibody providing the CDR's is called the “donor” and the human immunoglobulin providing the framework is called the “acceptor.” Humanization relies principally on the grafting of donor CDRs onto acceptor (human) VL and VH frameworks (Winter U.S. Pat. No. 5,225,539). This strategy is referred to as “CDR grafting”. “Backmutation” of selected acceptor framework residues to the corresponding donor residues is often required to regain affinity that is lost in the initial grafted construct (See e.g. U.S. Pat. No. 5,530,101; U.S. Pat. No. 5,585,089; U.S. Pat. No. 5,693,761; U.S. Pat. No. 5,693,762; U.S. Pat. No. 6,180,370; U.S. Pat. No. 5,859,205; U.S. Pat. No. 5,821,337; U.S. Pat. No. 6,054,297; U.S. Pat. No. 6,407,213). The humanized antibody optimally also will comprise at least a portion of an immunoglobulin constant region, typically that of a human immunoglobulin, and thus will typically comprise a human Fc region. Methods for humanizing non-human antibodies are well known in the art, and can be essentially performed following the method of Winter and co-workers (Jones et al., 1986, Nature 321:522-525; Riechmann et al., 1988, Nature 332:323-329; Verhoeyen et al., 1988, Science, 239:1534-1536). Additional examples of humanized murine monoclonal antibodies are also known in the art, for example antibodies binding human protein C(O'Connor et al., 1998, Protein Eng 11:321-8), interleukin 2 receptor (Queen et al., 1989, Proc Natl Acad Sci, USA 86:10029-33), and human epidermal growth factor receptor 2 (Carter et al., 1992, Proc Natl Acad Sci USA 89:4285-9). In some embodiments, the antibodies of the present invention may be fully human, that is the sequences of the antibodies are completely or substantially human. A number of methods are known in the art for generating fully human antibodies, including the use of transgenic mice (Bruggemann et al., 1997, Curr Opin Biotechnol 8:455-458) or human antibody libraries coupled with selection methods (Griffiths et al., 1998, Curr Opin Biotechnol 9:102-108).

Also included within the definition of “antibody” are aglycosylated antibodies. By “aglycosylated antibody” as used herein is meant an antibody that lacks carbohydrate attached at position 297 of the Fc region, wherein numbering is according to the EU system as in Kabat. The aglycosylated antibody may be a deglycosylated antibody, which is an antibody for which the Fc carbohydrate has been removed, for example chemically or enzymatically. Alternatively, the aglycosylated antibody may be a nonglycosylated or unglycosylated antibody, that is an antibody that was expressed without Fc carbohydrate, for example by mutation of one or more residues that encode the glycosylation pattern or by expression in an organism that does not attach carbohydrates to proteins, for example bacteria.

Also included within the definition of “antibody” are full-length antibodies that contain an Fc variant portion. By “full length antibody” herein is meant the structure that constitutes the natural biological form of an antibody, including variable and constant regions. For example, in most mammals, including humans and mice, the full length antibody of the IgG class is a tetramer and consists of two identical pairs of two immunoglobulin chains, each pair having one light and one heavy chain, each light chain comprising immunoglobulin domains VL and CL, and each heavy chain comprising immunoglobulin domains VH, Cg1, Cg2, and Cg3. In some mammals, for example in camels and llamas, IgG antibodies may consist of only two heavy chains, each heavy chain comprising a variable domain attached to the Fc region. By “IgG” as used herein is meant a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3.

In some embodiments, are immobilized on a substrate. Antibodies may be non-diffusibly bound to an insoluble support having isolated sample-receiving areas (e.g. a microtiter plate, an array, etc.). The insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes, and beads. These are typically made of glass, plastic (e.g., polystyrene), polysaccharides, nylon or nitrocellulose, Teflon™, etc. Microtiter plates and arrays are especially convenient because a large number of assays can be carried out simultaneously, using small amounts of reagents and samples. In some cases magnetic beads and the like are included.

In general, an antibody or other suitable binding partner is specific for a desired target antigen. By “specific” is meant a binding partner which is suitable for detection of the transcript or protein in a complex mixture. The binding partner may bind to the gene expression product preferentially over other transcripts/proteins in the same species and may have no or substantially no binding affinity for other proteins or transcripts. In the case of a transcript, the transcript is preferably capable of distinguishing the target transcript from other transcripts in the mixture at least under stringent hybridization conditions. In some embodiments, an antibody is specific to a particular LMCD1 variant (e.g. wild-type or a particular mutant protein), and binds the particular variant with greater affinity than other non-target LMCD1 variants. Accordingly, in one aspect, the invention provides an isolated antibody or antigen-binding antibody fragment thereof, directed specifically to a human LMCD1 mutant protein, or protein fragment thereof comprising one or more LMCD1 mutations. Examples of LMCD1 variants are described herein. In some embodiments, the epitope recognized by the isolated antibody or antigen-binding fragment thereof is predicted to be on the surface of the LMCD1 mutant protein. In some embodiments, the isolated antibody or antigen-binding fragment thereof binds an epitope in one or more of the PET domain or LIM domains of the mutant LMCD1 protein. In some embodiments, the affinity with which an antibody binds a particular LMCD1 variant is about or more than about 2, 3, 4, 5, 6, 7, 8, 9, 10, 15, 20, 25, 50, 75, 100, 150, 200, 300, 400, 500, 1000, 1×10⁴, 1×10⁵, 1×10⁶, 1×10⁷, 1×10⁸, or more fold greater than the affinity with which the antibody binds other, non-target LMCD1 variants.

Labels

Detection of an LMCD1 nucleic acid or protein through the use of a binding partner, such as an antibody or oligonucleotide probe, may be accomplished by any of a variety of methods known in the art. In some embodiments, the binding partner comprises a label. The term “label” is used to refer to a molecule that can be directly (i.e., a primary label) or indirectly (i.e., a secondary label) detected; for example a label can be visualized and/or measured or otherwise identified so that its presence or absence can be known. A compound can be directly or indirectly conjugated to a label which provides a detectable signal, e.g. radioisotopes, fluorescent moieties, enzymes, antibodies, particles such as magnetic particles, chemiluminescent moieties, or specific binding molecules, etc. Specific binding molecules include pairs, such as biotin and streptavidin, digoxin and antidigoxin etc. Preferred labels include, but are not limited to, optical fluorescent and chromogenic dyes including labels, label enzymes, and radioisotopes.

Labels include: isotopic labels, which may be radioactive or heavy isotopes; magnetic, electrical, and thermal labels; colored, optical labels including luminescent, phosphorous and fluorescent dyes or moieties; and binding partners. Labels can also include enzymes (horseradish peroxidase, etc.) and magnetic particles. In some embodiments, the detection label is a primary label. A primary label is one that can be directly detected, such as a fluorophore. Preferred labels include optical labels such as fluorescent dyes or moieties. Fluorophores include “small molecule” fluors, and proteinaceous fluors (e.g. green fluorescent proteins and all variants thereof).

The term “fluorescent label” encompasses any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, Texas Red, IAEDANS, EDANS, BODIPY FL, LC Red 640, Cy 5, Cy 5.5, LC Red 705 and Oregon green. Suitable optical dyes are described in the 1996 Molecular Probes Handbook by Richard P. Haugland, hereby expressly incorporated by reference.

Suitable fluorescent labels also include, but are not limited to, green fluorescent protein (GFP; Chalfie, et al., Science 263(5148):802-805 (Feb. 11, 1994); and EGFP; Clontech—Genbank Accession Number U55762), blue fluorescent protein (BFP; 1. Quantum Biotechnologies, Inc. 1801 de Maisonneuve Blvd. West, 8th Floor, Montreal (Quebec) Canada H3H 1J9; 2. Stauber, R. H. Biotechniques 24(3):462-471 (1998); 3. Heim, R. and Tsien, R. Y. Curr. Biol. 6:178-182 (1996)), enhanced yellow fluorescent protein (EYFP; 1. Clontech Laboratories, Inc., 1020 East Meadow Circle, Palo Alto, Calif. 94303), luciferase (Ichiki, et al., J. Immunol. 150(12):5408-5417 (1993)), β-galactosidase (Nolan, et al., Proc Natl Acad Sci USA 85(8):2603-2607 (April 1988)) and Renilla WO 92/15673; WO 95/07463; WO 98/14605; WO 98/26277; WO 99/49019; U.S. Pat. No. 5,292,658; U.S. Pat. No. 5,418,155; U.S. Pat. No. 5,683,888; U.S. Pat. No. 5,741,668; U.S. Pat. No. 5,777,079; U.S. Pat. No. 5,804,387; U.S. Pat. No. 5,874,304; U.S. Pat. No. 5,876,995; and U.S. Pat. No. 5,925,558). All of the above-cited references are expressly incorporated herein by reference.

Illustrative examples of useful labels include, but are not limited to: Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes) (Eugene, Oreg.), FITC, Rhodamine, and Texas Red (Pierce, Rockford, Ill.), Cy5, Cy5.5, Cy7 (Amersham Life Science, Pittsburgh, Pa.). In some embodiments, the label is a DNA-binding dye. Non-limiting examples of DNA-binding dyes suitable for this application include SYBR green, SYBR blue, DAPI, propidium iodine, Hoechst stain, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like. Further non-limiting examples of suitable labels include EvaGreen® (Biotium, Hayward, Calif.), SYBR® Green I, PicoGreen™, LC Green™, SYBR GreenER®, PO-PRO®.-1, BO-PRO®.-1, SYTO® 9, SYTO®™43, SYTO®. 44, SYTO®. 45, SYTOX® Blue, POPOT™.-1, POPO™.-3, BOBO™.-1, BOBO™-3, LO-PRO™-1, JO-PRO™-1, YO-PRO®-1, TO-PRO®-1, SYTO® 9, SYTO®11, SYTO®13, SYTO®15, SYTO®16, SYTO®20, SYTO®23, TOTO™3, YOYO®-3 (Molecular Probes, Inc., Eugene, Oreg.), GelStar® (Cambrex Bio Science Rockland Inc., Rockland, Me.), Ethidium Bromide, thiazole orange (Aldrich Chemical Co., Milwaukee, Wis.), BEBO, BETO, BOXTO (TATAA Biocenter AB., Goteborg, Sweden). Additional examples of labels are described in US20110136201, incorporated herein by reference. Tandem conjugate protocols for Cy5PE, Cy5.5PE, Cy7PE, Cy5.5APC, Cy7APC are known in the art. Quantitation of fluorescent probe conjugation may be assessed to determine degree of labeling and protocols including dye spectral properties are also well known in the art.

In some embodiments, a secondary detectable label is used. A secondary label is one that is indirectly detected; for example, a secondary label can bind or react with a primary label for detection, or can act on an additional product to generate a primary label (e.g. enzymes), etc. Secondary labels include, but are not limited to, one of a binding partner pair; chemically modifiable moieties; nuclease inhibitors; and enzymes such as horseradish peroxidase, alkaline phosphatases, lucifierases.

In some embodiments, the secondary label is a one of a pair of binding partners. For example, the label may be a hapten or antigen, which will bind its binding partner. For example, suitable binding partner pairs include, but are not limited to: antigens (such as proteins (including peptides) and small molecules) and antibodies (including fragments thereof (FAbs, etc.)); proteins and small molecules, including biotin/streptavidin; enzymes and substrates or inhibitors; other protein-protein interacting pairs; receptor-ligands; and carbohydrates and their binding partners. Nucleic acid-nucleic acid binding protein pairs are also useful. Preferred binding partner pairs include, but are not limited to, biotin (or imino-biotin) and streptavidin, digoxigenin and antibodies, and Prolinx™ reagents.

In some embodiments, the binding partner pair comprises an antigen and an antibody that will specifically bind to the antigen. The binding should be sufficient to remain bound under the conditions of the assay, including wash steps to remove non-specific binding. In some embodiments, the dissociation constants of the pair will be less than about 10⁻⁴ to 10⁻⁹M⁻¹, with less than about 10⁻⁵ to 10⁻⁹M⁻¹ being preferred and less than about 10⁻⁷ to 10⁻⁹M⁻¹ being particularly preferred.

Other possible labels include macromolecular colloidal particles or particulate material such as latex beads that are colored, magnetic or paramagnetic, and biologically or chemically active agents that can directly or indirectly cause detectable signals to be visually observed, electronically detected or otherwise recorded. Other methods may also be used to detect interaction between the protein and the antibody, including physical methods such as surface plasmon resonance, agglutination, light scattering or other means.

Therapeutic Compositions and Treatment

In one aspect, the invention provides a method of treating cancer in a subject. In one embodiment, the method comprises (a) providing a sample of a subject; (b) assaying said sample for the presence or absence of one or more LMCD1 mutations, wherein the presence of said one or more LMCD1 mutations indicates a higher propensity for metastasis than is indicated by the absence of said one or more LMCD1 mutations. In some embodiments, a method of the invention further comprises the step of selecting and optionally administering a therapeutic agent based on the results of determining the presence or absence of one or more LMCD1 mutations. In some embodiments, a method of the invention comprises step (c) treating said subject with an inhibitor that reduces metastasis, if said one or more LMCD1 mutations is present. Many therapeutic agents for the treatment of cancer are known in the art, and include without limitation chemotherapeutic agents, therapeutic antibodies, nucleic acids, polypeptides, small molecules, and radiation treatment.

In some embodiments, the inhibitor that recues metastasis is a chemotherapeutic agent. Many chemotherapeutic agents are known in the art, non-limiting examples of which include Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; emylerumines and memylamelamines including alfretamine, triemylenemelamine, triethylenephosphoramide, triethylenethiophosphoramide and trimemylolomelamine; acetogenins (especially bullatacin and bullatacinone); a camptothecin (including synthetic analogue topotecan); bryostatin; callystatin; CC-1065 (including its adozelesin, carzelesin and bizelesin synthetic analogues); cryptophycins (articularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, foremustine, lomustine, nimustine, ranimustine; antibiotics such as the enediyne antibiotics (e.g., calicheamicin, especially calicheamicin gammall and calicheamicin phili, see, e.g., Agnew, Chem. Intl. Ed. Engl, 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (Adramycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as demopterin, methotrexate, pteropterin, trimetrexate; purine analogs such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replinisher such as frolinic acid; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK™; razoxane; rhizoxin; sizofiran; spirogermanium; tenuazonic acid; triaziquone; 2,2′,2″-tricUorotriemylamine; trichothecenes (especially T-2 toxin, verracurin A, roridin A and anguidine); urethane; vindesine; dacarbazine; mannomustine; mitobronitol; mitolactol; pipobroman; gacytosine; arabinoside (“Ara-C”); cyclophosphamide; thiopeta; taxoids, e.g., paclitaxel (TAXOL™, Bristol Meyers Squibb Oncology, Princeton, N.J.) and docetaxel (TAXOTERET™., Rhone-Poulenc Rorer, Antony, France); chlorambucil; gemcitabine (Gemzar™); 6-thioguanine; mercaptopurine; methotrexate; platinum analogs such as cisplatin and carboplatin; vinblastine; platinum; etoposide (VP-16); ifosfamide; mitroxantrone; vancristine; vinorelbine (Navelbine™); novantrone; teniposide; edatrexate; daunomycin; aminopterin; xeoloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included in the definition of “chemotherapeutic agent” are anti-hormonal agents that act to regulate or inhibit hormone action on tumors such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston™); inhibitors of the enzyme aromatase, which regulates estrogen production in the adrenal glands, such as, for example, 4(5)-imidazoles, aminoglutethimide, megestrol acetate (Megace™), exemestane, formestane, fadrozole, vorozole (Rivisor™), letrozole (Femara™), and anastrozole (Arimidex™); and anti-androgens such as flutamide, nilutamide, bicalutamide, leuprohde, and goserelin; and pharmaceutically acceptable salts, acids or derivatives of any of the above. In some embodiments, the inhibitor downregulates Rac1 output. Additional examples of chemotherapeutic and other anti-cancer agents are described in US20090053224, incorporated herein by reference.

Decrease in propensity for metastasis is indicated by a decrease in cell migration. Many methods for measuring effects on cell migration are known in the art, and include, without limitation, trans-well migration assays, wound-healing assays, random migration assays, and morphological analysis for the presence and optionally number of lamellipodia. Methods for performing such assays are known in the art, the conditions of which may be optimized for a particular cell type, environment, or other relevant factor known to those skilled in the art. Examples of such assays are provided herein. In general, a decrease in cell migration, and corresponding decrease in propensity for metastasis, is indicated by a decreased number of migrated cells in a trans-well migration assay, a decrease in migration velocity of cells in a wound-healing assay, a decrease in migration velocity of cells in a random migration assay, and/or a decrease in the percentage of cells with lamellipodia. In some embodiments, treatment with an inhibitor of metastasis decreases the propensity for metastasis of a cell or tissue having an LMCD1 mutation by about or more than about 5%, 10%, 15%, 20%, 25%, 30%, 35%, 40%, 45%, 50%, 55%, 60%, 70%, 80%, 90%, 95%, 96%, 97%, 98%, 99%, or more compared to a similar untreated cell or tissue. In some embodiments, treatment with an inhibitor of metastasis decreases the propensity for metastasis of a cell or tissue having an LMC1 mutation by about or more than about 2-fold, 3-fold, 4-fold, 5-fold, 6-fold, 7-fold, 8-fold, 9-fold, 10-fold, 20-fold, 50-fold, 100-fold, 1000-fold, or more compared to a similar untreated cell or tissue.

In some embodiments, the therapeutic agent is an inhibitor that downregulates Rac1 output, such as an inhibitor of Rac1 or inhibitor of another component of the Rac1 pathway, such as LMCD1. Examples of Rac1 inhibitors include, without limitation, Rac1 Inhibitor W56, sold by Tocris Biosciences (Ellisville, Mo.), NSC23760 and NSC 23766 sold by EMD Biosciences (San Diego, Calif.), and the inhibitors described in Yuan Gao, et al. PNAS, May 18, 2004, vol. 101, 7618-7623. Other non-limiting examples of Rac1 inhibitors include F56, EHT 1864, and Rac1 Inhibitor II ((3-((3,5-Dimethylisoxazol-4-yl)methoxy)-N-(4-methyl-3-sulfamoyl-phenyl)benzamide, Z62954982). Rac1 Inhibitor W56 is a peptide comprising residues 45-60 of the guanine nucleotide exchange factor (GEF) recognition/activation site of Rac1; selectively inhibits Rac1 interaction with Rac1-specific GEFs TrioN, GEF-H1 and Tiam1 and is described in Gao et al (2001) J. Biol. Chem. 276 47530.

Downregulation of a target gene or gene transcript can be determined directly, such as by detecting the expression level of a gene expression product, such as an RNA transcript or protein product. Downregulation of a target gene or gene transcript can also be determined indirectly, such as by measuring the effect on a phenotypic indicator of the gene or gene transcript activity, such as by cellular assay. Methods of detecting gene expression products are known in the art, examples of which are described herein. In some embodiments, a decrease in Rac1 output is indicated by a decrease in the level of a Rac1 gene expression product by about or more than about 5%, 10%, 15%, 20%, 25%, 30%, 35%, 40%, 45%, 50%, 55%, 60%, 70%, 80%, 90%, 95%, 96%, 97%, 98%, 99%, or more compared to a similar untreated cell or tissue. In some embodiments, a decrease in Rac1 output is indicated by a decrease in cell migration. Many methods for measuring effects on cell migration are known in the art, and include, without limitation, trans-well migration assays, wound-healing assays, random migration assays, and morphological analysis for the presence and optionally number of lamellipodia. Methods for performing such assays are known in the art, the conditions of which may be optimized for a particular cell type, environment, or other relevant factor known to those skilled in the art. Examples of such assays are provided herein. In general, a decrease in cell migration, and corresponding decrease in Rac1 output, is indicated by a decreased number of migrated cells in a trans-well migration assay, a decrease in migration velocity of cells in a wound-healing assay, a decrease in migration velocity of cells in a random migration assay, and/or a decrease in the percentage of cells with lamellipodia. In some embodiments, treatment with an inhibitor that downregulates Rac1 output decreases Rac1 output of a cell or tissue having an LMCD1 mutation by about or more than about 5%, 10%, 15%, 20%, 25%, 30%, 35%, 40%, 45%, 50%, 55%, 60%, 70%, 80%, 90%, 95%, 96%, 97%, 98%, 99%, or more compared to a similar untreated cell or tissue. In some embodiments, treatment with an inhibitor that downregulates Rac1 output decreases Rac1 output of a cell or tissue having an LMCD1 mutation by about or more than about 2-fold, 3-fold, 4-fold, 5-fold, 6-fold, 7-fold, 8-fold, 9-fold, 10-fold, 20-fold, 50-fold, 100-fold, 1000-fold, or more compared to a similar untreated cell or tissue.

In some embodiments, treatment with the therapeutic agent decreases the likelihood that a cancer will metastasize, such as by about or more than about 5%, 10%, 15%, 20%, 25%, 30%, 35%, 40%, 45%, 50%, 55%, 60%, 70%, 80%, 90%, 95%, 96%, 97%, 98%, 99%, or more. In some embodiments, treatment with a therapeutic agent decreases the number of metastatic growths as compared with an untreated reference tissue (e.g. historic data relating to similar cancers), such as by about 5%, 10%, 15%, 20%, 25%, 30%, 35%, 40%, 45%, 50%, 55%, 60%, 70%, 80%, 90%, 95%, 96%, 97%, 98%, 99%, or more.

Nucleic acid therapeutics include nucleic acid molecules designed to trigger the RNA interference (RNAi) pathway for suppressing expression of one or more target transcripts. Non-limiting examples of RNAi triggers include shRNA, miRNA, siRNA, antisense RNA, and double-stranded RNA. RNAi may be used to create a pseudo “knockout”, i.e. a system in which the expression of the product encoded by a gene or coding region of interest is reduced, resulting in an overall reduction of the activity of the encoded product in a system. As such, RNAi may be performed to target a nucleic acid of interest or fragment or variant thereof, to in turn reduce its expression and the level of activity of the product which it encodes. Such a system may be used for functional studies of the product, as well as to treat disorders related to the activity of such a product. RNAi is described in for example Hammond et al. (2001) Science 10; 293(5532):1146-50., Caplen et al. (2001) Proc Natl Acad Sci USA. 2001 Aug. 14; 98(17):9742-7, all of which are herein incorporated by reference. Reagents and kits for performing RNAi are available commercially from for example Ambion Inc. (Austin, Tex., USA) and New England Biolabs Inc. (Beverly, Mass., USA).

The initial agent for RNAi in some systems is thought to be a dsRNA molecule corresponding to a target nucleic acid. The dsRNA is then thought to be cleaved into short interfering RNAs (siRNAs) which are 21-23 nucleotides in length (19-21 bp duplexes, each with 2 nucleotide 3′ overhangs). The enzyme thought to effect this first cleavage step has been referred to as “Dicer” and is categorized as a member of the RNase III family of dsRNA-specific ribonucleases. RNAi may also be effected via directly introducing into the cell, or generating within the cell by introducing into the cell a suitable precursor (e.g. vector encoding precursor(s), etc.) of such an siRNA or siRNA-like molecule. An siRNA may then associate with other intracellular components to form an RNA-induced silencing complex (RISC). The RISC thus formed may subsequently target a transcript of interest via base-pairing interactions between its siRNA component and the target transcript by virtue of homology, resulting in the cleavage of the target transcript approximately 12 nucleotides from the 3′ end of the siRNA. Thus the target mRNA is cleaved and the level of protein product it encodes is reduced. In some cases, inhibition is achieved without cleavage, such as is observed with many micro-RNAs (miRNAs).

RNAi may be effected by the introduction of suitable in vitro synthesized siRNA or siRNA-like molecules into cells. RNAi may for example be performed using chemically-synthesized RNA. Alternatively, suitable expression vectors may be used to transcribe such RNA either in vitro or in vivo. In vitro transcription of sense and antisense strands (encoded by sequences present on the same vector or on separate vectors) may be effected using for example T7 RNA polymerase, in which case the vector may comprise a suitable coding sequence operably-linked to a T7 promoter. The in vitro-transcribed RNA may in embodiments be processed (e.g. using E. coli RNase III) in vitro to a size conducive to RNAi. The sense and antisense transcripts are combined to form an RNA duplex which is introduced into a target cell of interest. Other vectors may be used, which express small hairpin RNAs (shRNAs) which can be processed into siRNA-like molecules. Various vector-based methods are described in for example Brummelkamp et al. (2002) Science April 19; 296(5567):550-3. Epub 2002 Mar. 21; Brummelkamp et al. Cancer Cell (2002) September 2(3):243-7. Paddison et al. (2002) Genes Dev. 2002 Apr. 15; 16(8):948-58. Various methods for introducing such vectors into cells, either in vitro or in vivo (e.g. gene therapy) are known in the art.

Accordingly, in an embodiment the expression of one or more genes or gene expression products in the Rac1 pathway may be inhibited by introducing into or generating within a cell an siRNA or siRNA-like molecule corresponding to a nucleic acid encoding the target gene or fragment thereof, or to an nucleic acid homologous thereto. “siRNA-like molecule” refers to a nucleic acid molecule similar to an siRNA (e.g. in size and structure) and capable of eliciting siRNA or miRNA activity, i.e. to effect the RNAi-mediated inhibition of expression. In various embodiments such a method may entail the direct administration of the siRNA or siRNA-like molecule into a cell, or use of the vector-based methods described above. In an embodiment, the siRNA or siRNA-like molecule is less than about 30 nucleotides in length. In a further embodiment, the siRNA or siRNA-like molecule is about 21-23 nucleotides in length. In an embodiment, siRNA or siRNA-like molecule comprises a 19-21 bp duplex portion, each strand having a 2 nucleotide 3′ overhang. In some embodiments, the siRNA or siRNA-like molecule is substantially homologous to a nucleic acid encoding the target gene or a fragment or variant (or a fragment of a variant) thereof. In some embodiments, the target gene is one or more of LMCD1 or Rac1.

Various delivery systems are known and can be used to administer a biologically active agent of the invention, e.g., encapsulation in liposomes, microparticles, microcapsules, expression by recombinant cells, receptor-mediated endocytosis (see, e.g., Wu and Wu, (1987), J. Biol. Chem. 262:4429-4432), construction of a therapeutic nucleic acid as part of a retroviral or other vector, etc. Methods of delivery include but are not limited to intra-arterial, intra-muscular, intravenous, intranasal, and oral routes. In a specific embodiment, it may be desirable to administer the pharmaceutical compositions of the invention locally to the area in need of treatment; this may be achieved by, for example, and not by way of limitation, local infusion during surgery, by injection, or by means of a catheter.

Administration of the selected agent can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician.

The preparation of pharmaceutical compositions of this invention is conducted in accordance with generally accepted procedures for the preparation of pharmaceutical preparations. See, for example, Remington's Pharmaceutical Sciences 18th Edition (1990), E. W. Martin ed., Mack Publishing Co., PA. Depending on the intended use and mode of administration, it may be desirable to process the active ingredient further in the preparation of pharmaceutical compositions. Appropriate processing may include mixing with appropriate non-toxic and non-interfering components, sterilizing, dividing into dose units, and enclosing in a delivery device.

Pharmaceutical compositions for oral, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or aerosol when used with an appropriate aerosolizer device.

Liquid pharmaceutically acceptable compositions can, for example, be prepared by dissolving or dispersing a polypeptide embodied herein in a liquid excipient, such as water, saline, aqueous dextrose, glycerol, or ethanol. The composition can also contain other medicinal agents, pharmaceutical agents, adjuvants, carriers, and auxiliary substances such as wetting or emulsifying agents, and pH buffering agents.

Where desired, the pharmaceutical compositions can be formulated in slow release or sustained release forms, whereby a relatively consistent level of the active compound are provided over an extended period.

Screening Inhibitors

In one aspect, the invention provides a method for screening inhibitors of cancer metastasis. In one embodiments, the method comprises (a) providing a cell line expressing LMCD1 having one or more LMCD1 mutations; (b) exposing said cell line to a test compound; (c) determining the effect of said compound on cell migration, wherein a decrease in cell migration of cells treated with said compound compared to cells not treated with said compound identifies said compound as an inhibitor of cancer metastasis. The LMCD1 having one or more LMCD1 mutations may be an endogenously arising LMCD1, or the LMCD1 may be expressed from a transgene. The transgene may be stably integrated, or expressed extrachromasomally, such as from a plasmid or viral genome. Examples of LMCD1 mutations are provided herein. Test compounds include any agent to which a test cell may be exposed, including but not limited to polypeptides, nucleic acids, small organic molecules, small inorganic molecules, ligands, aptamers, antibodies, radiation, and light of one or more selected frequencies. In some embodiments, the test compound is a nucleic acid trigger that triggers the RNAi pathway, such as an shRNA, an miRNA, an siRNA, an antisense RNA, and a double-stranded RNA.

Methods for measuring effects on cell migration are known in the art, and include, without limitation, trans-well migration assays, wound-healing assays, random migration assays, and morphological analysis for the presence and optionally number of lamellipodia. Methods for performing such assays are known in the art, the conditions of which may be optimized for a particular cell type, environment, or other relevant factor known to those skilled in the art. Examples of such assays are provided herein. In general, a decrease in cell migration, is indicated by a decreased number of migrated cells in a trans-well migration assay, a decrease in migration velocity of cells in a wound-healing assay, a decrease in migration velocity of cells in a random migration assay, and/or a decrease in the percentage of cells with lamellipodia. In some embodiments, a test compound is identified as an inhibitor of metastasis when treatment with the compound decreases a measure of cell migration of a cell or tissue having an LMCD1 mutation by about or more than about 5%, 10%, 15%, 20%, 25%, 30%, 35%, 40%, 45%, 50%, 55%, 60%, 70%, 80%, 90%, 95%, 96%, 97%, 98%, 99%, or more compared to a similar untreated cell or tissue. In some embodiments, a test compound is identified as an inhibitor of metastasis when treatment with the compound decreases a measure of cell migration of a cell or tissue having an LMCD1 mutation by about or more than about 2-fold, 3-fold, 4-fold, 5-fold, 6-fold, 7-fold, 8-fold, 9-fold, 10-fold, 20-fold, 50-fold, 100-fold, 1000-fold, or more compared to a similar untreated cell or tissue.

In some embodiments, the effects on cell migration are assayed by detecting migration of treated cells compared to untreated cells when transplanted into a non-human animal, such as a mouse, rat, rabbit, dog, or non-human primate. Transplantation may be by any suitable method, such as by injection or surgical implantation. In some embodiments, transplanted cells express a marker, such as a visible marker or detectable enzyme activity, by which transplanted cells may be subsequently identified in tissues into which transplanted cells may have migrated. In one embodiment, cells expressing LMCD1 having one or more LMCD1 mutations are transplanted before exposure to the test compound, which is subsequently administered to the non-human animal. In another embodiment, cells expressing LMCD1 having one or more LMCD1 mutations are transplanted after exposure to the test compound, such as in culture. When cells are exposed prior to transplantation, exposure may optionally be continued by administering the test compound to the non-human animal after transplantation. In general, a decrease in cell migration is indicated by a decrease in the number of said cells found in a tissue that is not the tissue into which said cells were transplanted. For example, cells may be transplanted by injection into the circulation, and one or more body organs examined for the presence of migrated cells. In some embodiments, a test compound is identified as an inhibitor of metastasis when treatment with the test compound decreases cell migration to one or more tissues into which cells were not transplanted by about, or more than about 5%, 10%, 15%, 20%, 25%, 30%, 35%, 40%, 45%, 50%, 55%, 60%, 70%, 80%, 90%, 95%, 96%, 97%, 98%, 99%, or more compared to untreated cells. In some embodiments, a test compound is identified as an inhibitor of metastasis when treatment with the test compound decreases cell migration to one or more tissues into which cells were not transplanted by about, or more than about 2-fold, 3-fold, 4-fold, 5-fold, 6-fold, 7-fold, 8-fold, 9-fold, 10-fold, 20-fold, 50-fold, 100-fold, 1000-fold, or more compared to untreated cells.

In some embodiments, transgenic animals are used instead of transplanted cells for screening one or more test compounds for an ability to inhibit cancer metastasis, such as metastasis associate with the expression of one or more LMCD1 mutations. In some embodiments, the method comprises (a) providing a transgenic non-human animal comprising cancer cells expressing LMCD1 having one or more LMCD1 mutations; (b) administering to the transgenic non-human animal a test compounds; and (c) determining the effect of the test compounds on cancer metastasis, wherein a decrease in metastasis of the cancer cells in the treated non-human transgenic animal as compared to an untreated non-human transgenic animal identifies the test compounds as an inhibitor of cancer metastasis. A decrease in metastasis may be determined by assaying for a decrease in the number of metastatic growths in a tissue from which cancer cells expressing LMCD1 did not originate. Methods for determining tissue of origin are known in the art and include, without limitation, morphological analysis and detection of one or more cell-type markers. In some embodiments, a test compound is identified as an inhibitor of metastasis when treatment with the test compound decreases the number of metastatic growths in tissues from which the cancer cells expressing the mutant LMCD1 did not originate by about, or more than about 5%, 10%, 15%, 20%, 25%, 30%, 35%, 40%, 45%, 50%, 55%, 60%, 70%, 80%, 90%, 95%, 96%, 97%, 98%, 99%, or more compared to cancer cells expressing the mutant LMCD1 in an untreated animal. In some embodiments, a test compound is identified as an inhibitor of metastasis when treatment with the test compound decreases the number of metastatic growths in tissues from which the cancer cells expressing the mutant LMCD1 did not originate by about, or more than about 2-fold, 3-fold, 4-fold, 5-fold, 6-fold, 7-fold, 8-fold, 9-fold, 10-fold, 20-fold, 50-fold, 100-fold, 1000-fold, or more compared to cancer cells expressing the mutant LMCD1 in an untreated animal.

Expression Vectors

In one aspect, the invention provides expression vectors for the expression of an LMCD1 mutant protein, and host cells comprising the same. In one embodiment, the expression vector comprises a nucleotide sequence encoding a mutant LMCD1 comprising one or more LMCD1 mutations, wherein expression of said mutant LMCD1 increases cell mobility in cells that actively express said expression vector. Examples of LMCD1 mutations are provided herein, and include without limitation G517A, A824G, E135K, K237R, and R154C. Methods for measuring effects on cell mobility are known in the art, and include, without limitation, trans-well migration assays, wound-healing assays, random migration assays, and morphological analysis for the presence and optionally number of lamellipodia. Methods for performing such assays are known in the art, the conditions of which may be optimized for a particular cell type, environment, or other relevant factor known to those skilled in the art. Examples of such assays are provided herein. In general, an increase in cell mobility is indicated by an increased number of migrated cells in a trans-well migration assay, an increase in migration velocity of cells in a wound-healing assay, an increase in migration velocity of cells in a random migration assay, and/or an increase in the percentage of cells with lamellipodia. In some embodiments, expression of the mutant LMCD1 increases a measure of cell mobility by about or more than about 5%, 10%, 15%, 20%, 25%, 30%, 35%, 40%, 45%, 50%, 55%, 60%, 70%, 80%, 90%, 95%, 96%, 97%, 98%, 99%, or more compared to a similar cell or tissue not expressing the mutant LMCD1. In some embodiments, expression of the mutant LMCD1 increases a measure of cell mobility by about or more than about 2-fold, 3-fold, 4-fold, 5-fold, 6-fold, 7-fold, 8-fold, 9-fold, 10-fold, 20-fold, 50-fold, 100-fold, 1000-fold, or more compared to a similar cell or tissue not expressing the mutant LMCD1.

For recombinant expression of an LMCD1 mutant protein, the nucleic acid encoding it is isolated and inserted into a replicable vector for further cloning (amplification of the DNA) or for expression. In some embodiments, wild-type LMCD1 is cloned into the vector, and the wild-type sequence is mutated to produce a mutant LMCD1 expression vector, such as by directed mutation using methods known in the art (e.g. by PCR with a primer containing the desired mutation). DNA encoding wild-type and mutant LMCD1 is readily isolated and sequenced using conventional procedures (e.g., by using oligonucleotide probes and primers). Many vectors are available. The vector components generally include, but are not limited to, one or more of the following: a signal sequence, an origin of replication, one or more marker genes, an enhancer element, a promoter, and a transcription-termination sequence.

In general, and unless the expression vector is introduced into a host cell chromosome, both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid, G418, kanamycin, and hygromycin. See U.S. Pat. No. 4,965,199.

1001481A suitable selection gene for use in yeast is the trp1 gene present in the yeast plasmid YRp7 (Stinchcomb et al., Nature, 282:39 (1979)). The trp1 gene provides a selection marker for a mutant strain of yeast lacking the ability to grow in tryptophan, for example, ATCC No. 44076 or PEP4-1. Jones, Genetics, 85:12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene. In addition, vectors derived from the 1.6-μm circular plasmid pKD1 can be used for transformation of Kluyveromyces yeasts. Alternatively, an expression system for large-scale production of recombinant calf chymosin was reported for K. lactis. Van den Berg, Bio/Technology, 8:135 (1990). Stable multi-copy expression vectors for secretion of mature recombinant human serum albumin by industrial strains of Kluyveromyces have also been disclosed. Fleer et al., Bio/Technology, 2: 968-975 (1991).

In some embodiments, the expression vector also comprises a nucleotide sequence encoding a detectable label. A detectable label may include, but is not limited to an enzyme, a transcription factor, a radioisotope binding protein, a fluorescent protein, or a fluorescent protein complex. In certain aspects, the fluorescent protein is a green fluorescent protein (GFP), cyan fluorescent protein (CFP), blue fluorescent protein (BFP), yellow fluorescent protein (YFP), red fluorescent protein (RFP), variants thereof, or various combinations thereof. In some embodiments, the detectable label is detectable by fluorescence, enzymatic activity, FRET, or NMR.

Expression and cloning vectors usually contain a promoter that is recognized by the host organism and is operably linked to the LMCD1 mutant protein-encoding nucleic acid. Promoters suitable for use with prokaryotic hosts include the phoA promoter, β-lactamase and lactose promoter systems, alkaline phosphatase, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the LMCD1 mutant protein.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with eukaryotic hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other eukaryotic promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in eukaryotic expression are further described in EP 73,657. Eukaryotic enhancers also are advantageously used with eukaryotic promoters. Non-limiting examples of eukaryotic cells include yeast cells and mammalian cell lines.

LMCD1 mutant protein expression from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), heterologous mammalian promoters, e.g., the actin promoter or an immunoglobulin promoter, and heat-shock promoters, provided such promoters are compatible with the host cell systems. The early and late promoters of the SV40 virus are conveniently obtained as an SV40 restriction fragment that also contains the SV40 viral origin of replication. The immediate early promoter of the human cytomegalovirus is conveniently obtained as a Hin III E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature, 297:598-601 (1982) on expression of human β-interferon cDNA in mouse cells under the control of a thymidine kinase promoter from herpes simplex virus. Alternatively, the rous sarcoma virus long-terminal repeat can be used as the promoter.

Transcription of a DNA encoding an LMCD1 mutant protein by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early-promoter enhancer, the polyoma enhancer on the late side of the replication origin, and adenovirus enhancers. See also Yaniv, Nature, 297:17-18 (1982) on enhancing elements for activation of eukaryotic promoters. The enhancer may be spliced into the vector at a position 5′ or 3′ to the LMCD1 mutant protein-encoding sequence, but is preferably located at a site 5′ from the promoter.

Expression vectors used in eukaryotic host cells (for example, yeast, fungi, insect, plant, animal, human, or nucleated cells from other multicellular organisms) will also contain sequences necessary for the termination of transcription and for stabilizing the mRNA. Such sequences are commonly available from the 5′ end, occasionally 3′ end, of untranslated regions of eukaryotic or viral DNAs or cDNAs. These regions contain nucleotide segments transcribed as polyadenylated fragments in the untranslated portion of the mRNA encoding LMCD1 mutant protein. One useful transcription termination component is the bovine growth hormone polyadenylation region. See WO 1994/11026 and the expression vector disclosed therein.

Host Cells

In one aspect, the invention provides a transgenic cell comprising an expression vector comprising a nucleotide sequence encoding a mutant LMCD1 comprising one or more LMCD1 mutations, such as expression vectors described herein. In some embodiments, the expression vector is extrachromosomal, such as a plasmid. In some embodiments, the transgenic cell comprises a stably integrated transgenic nucleotide sequence encoding a mutant LMCD1 comprising one or more LMCD1 mutations. Under suitable conditions, the transgenic cell actively expresses the mutant LMCD1 and exhibits increased cell mobility relative to a cell not expressing the mutant LMCD1. Conditions suitable for expression depend on a number of factors known in the art, such as growth conditions for the cells and the activity of the promoter driving expression, which may be constitutively active, active is specific cell types, inducible in response to the presence of an inducing agent, or any other promoter described herein or known in the art. Examples of LMCD1 mutations are provided herein, and include without limitation G517A, A824G, E135K, K237R, and R154C. Methods for measuring effects on cell mobility are known in the art, and include, without limitation, trans-well migration assays, wound-healing assays, random migration assays, and morphological analysis for the presence and optionally number of lamellipodia. Methods for performing such assays are known in the art, the conditions of which may be optimized for a particular cell type, environment, or other relevant factor known to those skilled in the art. Examples of such assays are provided herein. In general, an increase in cell mobility is indicated by an increased number of migrated cells in a trans-well migration assay, an increase in migration velocity of cells in a wound-healing assay, an increase in migration velocity of cells in a random migration assay, and/or an increase in the percentage of cells with lamellipodia. In some embodiments, expression of the mutant LMCD1 increases a measure of cell mobility by about or more than about 5%, 10%, 15%, 20%, 25%, 30%, 35%, 40%, 45%, 50%, 55%, 60%, 70%, 80%, 90%, 95%, 96%, 97%, 98%, 99%, or more compared to a similar cell or tissue not expressing the mutant LMCD1. In some embodiments, expression of the mutant LMCD1 increases a measure of cell mobility by about or more than about 2-fold, 3-fold, 4-fold, 5-fold, 6-fold, 7-fold, 8-fold, 9-fold, 10-fold, 20-fold, 50-fold, 100-fold, 1000-fold, or more compared to a similar cell or tissue not expressing the mutant LMCD1.

In some embodiments the transgenic cell expresses a selectable marker. Examples of selectable markers are provided herein, and may be expressed from the expression vector encoding the mutant LMCD1, or separately, such as from another expression vector which may or may not be integrated into the host cell genome. In some embodiments, the transgenic cell expresses a detectable label. Examples of detectable labels are provided herein, and may be expressed from the expression vector encoding the mutant LMCD1, or separately, such as from another expression vector which may or may not be integrated into the host cell genome.

Suitable host cells for cloning or expressing the DNA in the vectors herein are the prokaryote, yeast, or higher eukaryote cells described herein and otherwise known in the art. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for LMCD1 mutant protein-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of mutant LMCD1 include cells derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells/−DHFR (CHO, Urlaub et al., Proc. Natl. Acad. Sci. USA, 77:4216 (1980), including DG44 (Urlaub et al., Som. Cell and Mol. Gen., 12: 555-566 (1986)) and DP12 cell lines); mouse sertoli cells (TM4, Mather, Biol. Reprod., 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci., 383:44-68 (1982)); MRC 5 cells; FS4 cells; and a human hepatoma line (Hep G2).

A wide variety of additional cell lines for various tissue culture applications, gene expression, and assays are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassus, Va.)).

Host cells can be transfected with one or more of the above-described expression or cloning vectors for anti LMCD1 mutant protein expression and cultured in conventional nutrient media modified as appropriate for inducing promoters, selecting transformants, or amplifying the genes encoding the desired sequences.

Host cells transfected with expression vectors for the expression of an LMCD1 mutant protein may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described, for example, in Ham et al., Meth. Enz. 58:44 (1979); Barnes et al., Anal. Biochem. 102:255 (1980); U.S. Pat. No. 4,767,704; 4,657,866; 4,927,762; 4,560,655; or 5,122,469; WO 1990/03430; WO 1987/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Several transfection protocols are known in the art, and are reviewed in Kaufman R. J., et al., Nucleic Acids Res. 19:4485, 1991. The transfection protocol chosen will depend on the host cell type and the nature of the expression vector, and can be chosen based upon routine experimentation. Typically, a transfection protocol includes introducing an expression vector into a suitable host cell, and then identifying and isolating host cells which have incorporated the heterologous DNA in a stable, expressible manner.

One commonly used method of introducing heterologous DNA is calcium phosphate precipitation, for example, as described by Wigler et al. (Proc. Natl. Acad. Sci. USA 77:3567, 1980). DNA introduced into a host cell by this method frequently undergoes rearrangement, making this procedure useful for cotransfection of independent genes.

Polyethylene-induced fusion of bacterial protoplasts with mammalian cells (Schaffner et al., Proc. Natl. Acad. Sci. USA 77:2163, 1980) is another useful method of introducing heterologous DNA. Protoplast fusion protocols frequently yield multiple copies of the plasmid DNA integrated into the mammalian host cell genome. This technique typically requires the selection and amplification marker to be on the same plasmid as the gene of interest.

Electroporation can also be used to introduce DNA directly into the cytoplasm of a host cell, as described by Potter et al. (Proc. Natl. Acad. Sci. USA 81:7161, 1988) or Shigekawa and Dower (BioTechniques 6:742, 1988). In general, electroporation does not require the selection marker and the gene of interest to be on the same plasmid.

Several reagents useful for introducing heterologous DNA into a mammalian cell have been described. These include Lipofectin® Reagent and Lipofectamine™ Reagent (Gibco BRL, Gaithersburg, Md.). Both of these reagents are commercially available reagents used to form lipid-nucleic acid complexes (or liposomes) which, when applied to cultured cells, facilitate uptake of the nucleic acid into the cells.

Transfection of cells with heterologous DNA and selection for cells that have taken up the heterologous DNA and express the selectable marker results in a pool of transfected cells. Individual cells in these pools may vary in the amount of DNA incorporated and in the chromosomal location of the transfected DNA. After repeated passage, pools may lose the ability to express the heterologous protein. To generate stable cell lines, individual cells can be isolated from the pools and cultured (a process referred to as cloning). In some instances, the pools themselves may be stable (e.g., production of the heterologous recombinant protein remains stable). The ability to select and culture such stable pools of cells would be desirable as it would allow rapid production of relatively large amounts of recombinant protein from mammalian cells.

In some embodiments, the expression vector is packaged within a virus, which is then used to transfect cells. Examples of suitable viruses, and accompanying packaging and delivery methods are known in the art.

Transgenic Animals

In one aspect, the invention provides a non-human transgenic animal whose genome comprises a stably integrated nucleotide sequence encoding a mutant LMCD1 comprising one or more LMCD1 mutations. In one embodiment, cancer cells arising from the cells of the non-human transgenic animal and expressing the mutant LMCD1 exhibit a higher propensity for metastasis relative to cancer cells not expressing the mutant LMCD1. Conditions suitable for expression depends on a number of factors known in the art, such as the activity of the promoter driving expression, which may be constitutively active, active is specific cell types, inducible in response to the presence of an inducing agent, or any other promoter described herein or known in the art. Examples of LMCD1 mutations are provided herein, and include without limitation G517A, A824G, E135K, K237R, and R154C. Methods for determining effects on the propensity for cells to metastasize are known in the art, and include methods for measuring effects on cell mobility. Methods for measuring effects on cell mobility are known in the art, and include, without limitation, trans-well migration assays, wound-healing assays, random migration assays, and morphological analysis for the presence and optionally number of lamellipodia. Methods for performing such assays are known in the art, the conditions of which may be optimized for a particular cell type, environment, or other relevant factor known to those skilled in the art. Examples of such assays are provided herein. In general, an increase in cell mobility is indicated by an increased number of migrated cells in a trans-well migration assay, an increase in migration velocity of cells in a wound-healing assay, an increase in migration velocity of cells in a random migration assay, and/or an increase in the percentage of cells with lamellipodia. In some embodiments, expression of the mutant LMCD1 increases a measure of cell mobility by about or more than about 5%, 10%, 15%, 20%, 25%, 30%, 35%, 40%, 45%, 50%, 55%, 60%, 70%, 80%, 90%, 95%, 96%, 97%, 98%, 99%, or more compared to a similar cell or tissue not expressing the mutant LMCD1. In some embodiments, expression of the mutant LMCD1 increases a measure of cell mobility by about or more than about 2-fold, 3-fold, 4-fold, 5-fold, 6-fold, 7-fold, 8-fold, 9-fold, 10-fold, 20-fold, 50-fold, 100-fold, 1000-fold, or more compared to a similar cell or tissue not expressing the mutant LMCD1. An increase in propensity for metastasis may also be determined by assaying for an increase in the number of metastatic growths in a tissue from which cells expressing LMCD1 did not originate. Methods for determining tissue of origin are known in the art and include, without limitation, morphological analysis and detection of one or more cell-type markers. In general, an increase in the number of metastatic growths is measured relative to some reference population of cells, such as a similar cancer in a similar animal not expressing the mutant LMCD1.

In some embodiments, a higher propensity for metastasis indicates a likelihood of metastasis of a cell or tissue of the transgenic animal expressing one or more LMCD1 mutations that is about or more than about 5%, 10%, 15%, 20%, 25%, 30%, 35%, 40%, 45%, 50%, 55%, 60%, 70%, 80%, 90%, 100%, or higher than the likelihood of metastasis of a reference cell or tissue lacking the one or more LMCD1 mutations. In some embodiments, a higher propensity for metastasis of a cell or tissue of the transgenic animal expressing one or more LMCD1 mutations indicates a likelihood of metastasis that is about or more than about 2-fold, 3-fold, 4-fold, 5-fold, 6-fold, 7-fold, 8-fold, 9-fold, 10-fold, 20-fold, 50-fold, 100-fold, 1000-fold, or more than a reference cell or tissue lacking the one or more LMCD1 mutations.

The present invention contemplates transgenic animals that carry one or more desired transgenes in all their cells, as well as animals which carry the transgenes in some, but not all their cells, i.e., mosaic animals. Animals of any species, including, but not limited to, mice, rats, rabbits, guinea pigs, pigs, micro-pigs, goats, and non-human primates, e.g., baboons, monkeys, and chimpanzees may be used to generate the subject transgenic animals. In some embodiments, cells of the transgenic animal express a selectable marker. Examples of selectable markers are provided herein, and may be expressed from the expression vector encoding the mutant LMCD1, or separately, such as from another expression vector integrated into the genome of the transgenic animal. In some embodiments, cells of the transgenic animal express a detectable label. Examples of detectable labels are provided herein, and may be expressed from the expression vector encoding the mutant LMCD1, or separately, such as from another expression vector integrated into the genome of the transgenic animal.

A desired transgene may be integrated as a single copy or in concatamers, e.g., head-to-head tandems or head-to-tail tandems. The desired transgene may also be selectively introduced into and activated in a particular tissue or cell type, preferably cells within the central nervous system. The regulatory sequences required for such a cell-type specific activation will depend upon the particular cell type of interest, and will be apparent to those of skill in the art. Where the animal to be made transgenic carries an endogenous LMCD1 gene, the transgene may be integrated elsewhere as an additional copy, or integrated at the site of the endogenous copy so as to replace it.

When it is desired that the transgene be integrated into the chromosomal site of the endogenous counterpart, gene targeting is preferred. Briefly, when such a technique is to be utilized, vectors containing some nucleotide sequences homologous to the endogenous counterpart are designed for the purpose of integrating, via homologous recombination with chromosomal sequences, into and disrupting the function of the nucleotide sequence of the endogenous gene.

Advances in technologies for embryo micromanipulation now permit introduction of heterologous DNA into fertilized mammalian ova as well. For instance, totipotent or pluripotent stem cells can be transformed by microinjection, calcium phosphate mediated precipitation, liposome fusion, retroviral infection or other means. The transformed cells are then introduced into the embryo, and the embryo will then develop into a transgenic animal. In some embodiments, developing embryos are infected with a viral vector containing a desired transgene so that the transgenic animals expressing the transgene can be produced from the infected embryo. In some embodiments, a desired transgene is coinjected into the pronucleus or cytoplasm of the embryo, preferably at the single cell stage, and the embryo is allowed to develop into a mature transgenic animal. These and other variant methods for generating transgenic animals are well established in the art and hence are not detailed herein. See, for example, U.S. Pat. Nos. 5,175,385 and 5,175,384.

In some embodiments, cells derived from such transgenic animals are used for conducting cell-based assays for screening and developing agents effective for inhibiting metastasis. In some embodiments, transgenic animals are used to screen one or more test compounds to identify inhibitors of metastasis, such as described above.

Kits

In one aspect, the invention provides kits containing any one or more of the elements disclosed in the above methods and compositions. In some embodiments, a kit comprises a composition of the invention, in one or more containers. A kit of the invention may comprise one or more compositions of the invention and instructions instructing the use of said composition. For example, a kit may comprise one or more of the following: reagents suitable for detecting a mutant LMCD1, such as an oligonucleotide or a protein binding element; one or more buffers for a detection reaction; a protocol for carrying out an assay; optionally any additional reagents; and optionally any reference standard. In some embodiments, a kit comprises one or more of the following: an expression vector and/or transgenic cell; one or more buffers; one or more standards; a protocol for the use of said expression vector and/or transgenic cell; and optionally any other reagents for an assay.

Reporting Results

In some embodiments, a method of the invention comprises reporting results of an assay. Reporting may be by any manner of communication known in the art. Communication may local to where the results were generated, or may be communicated to another location, such as by wired or wireless communication. Non-limiting examples of wireless communication include bluetooth, RTM technology, or wireless internet connection. Various communication methods can be utilized, such as a dial-up wired connection with a modem, a direct link such as a T1, ISDN, or cable line. In some embodiments, a wireless connection is established using exemplary wireless networks such as cellular, satellite, or pager networks, or a local data transport system such as Ethernet or token ring over a local area network. In some embodiments, the information is encrypted before it is transmitted over a wireless network. In some embodiments, the communication assembly may contain a wireless infrared communication component for sending and receiving information. In some embodiments, results are reported to a particular person or entity. Non-limiting examples of persons or entities to whom results may be reported include a patient, medical personnel, clinicians, laboratory personnel, insurance company personnel, or others in the health care industry.

In some embodiments, results are communicated to an external device. In some embodiments the external device can be a computer system, server, PDA, cell phone, or other electronic device capable of storing information or processing information. In some embodiments the external device includes one or more computer systems, servers, or other electronic devices capable of storing information or processing information. In some embodiments an external device may include a database of patient information, for example but not limited to, medical records or patient history, clinical trial records, or preclinical trial records. A server can include a database and system processes. A database can reside within the server, or it can reside on another server system that is accessible to the server. As the information in a database may contain sensitive information, a security system can be implemented that prevents unauthorized users from gaining access to the database.

Sequences
SEQ ID NO: 1
LMCD1 mRNA sequence (NM_014583.2)
acagagctccctcccaggcccgcgaacttggccattcagccgccgctgtccccgctgcgcgccctcgcgcctctgcctgagaagccaggcgctgttccccca
ccccagaagaggatggcaaaggtggctaaggacctcaacccaggagttaaaaagatgtccctgggccagctgcagtcagcaagaggtgtggcatgtttggg
atgcaaggggacgtgttcgggcttcgagccacattcatggaggaaaatatgcaagtcttgcaaatgcagccaagaggaccactgcctaacatctgacctagaa
gacgatcggaaaattggccgcttgctgatggactccaagtattccaccctcactgctcgggtgaaaggcggggacggcatccggatttacaagaggaaccgg
atgatcatgaccaaccctattgctactgggaaagatcccacttttgacaccatcacctacgagtgggctccccctggagtcacccagaaactgggactgcagta
catgGagctcatccccaaggagaagcagccagtgacaggcacagagggtgccttttaccgccgccgccagctcatgcaccagctccccatctatgaccagg
atccctcgcgctgccgtggacttttggagaatgagttgaaactgatggaagaatttgtcaagcaatataagagcgaggccctcggcgtgggagaagtggccct
cccggggcagggtggcttgcccaaggaggaggggaagcagcaggaaaagccagagggggcagagaccactgctgctaccaccaacggcagtctcagtg
acccgtccaAagaagtggaatacgtctgcgagctctgcaagggagcggcccctcctgacagccccgtggtctactcggacagggcaggctacaacaagca
gtggcaccccacctgctttgtgtgtgccaagtgctccgagccgctggtggacctcatctacttctggaaggatggtgcaccctggtgcggccgccattactgcg
agagtctgcggccccggtgctccggctgcgatgagataatattcgctgaggactaccagcgtgtggaagatctggcctggcaccgaaagcactttgtctgtga
gggttgtgagcagctgctgagcggccgggcgtacatcgtcaccaagggtcagcttctgtgcccaacttgcagcaagtccaaacgctcctgaagggctgccca
cccacagccagaatccacaggatcccaccgagaaggagagccaggtgtgccgagaccatcctaagggtccgatgtgacagcaagcaagtgaaataaacaa
tgatttgcttttcagtgagaatatatatatgagatatatatagatatatattctaggttgggtggtggtagatccttgagggtcagtagtttcaaaaccaaaaa
tattctaagaagtcttaggatggagttccttttctttctgttgttgtttcccagctacaaccaactaaagacacaaatggcgttctgcaaggggactctgggag
gagttttccagaatgcaattccgagtgagcaaatcgcatagctgtagaatgtgcgtgcttttttgtggacacaggagctcctccaggagcaggctgggatccca
actatcgcttgttgcctctttttcaagtggaatttgaattttaaataaacaactttttttggcatgataaacagatcaataaaagttttgtgaattccaaaaaa
aaaaaaaaaaaaaaa
SEQ ID NO: 2
LMCD1 protein sequence (NP_055398; 365 aa)
makvakdlnpgvkkmslgqlqsargvaclgckgtcsgfephswrkicksckcsqedhcltsdleddrkigrllmdskystltarvkggdgiriykrnrmi
mtnpiatgkdptfdtityewappgvtqklglqymElipkekqpvtgtegafyrrrqlmhqlpiydqdpsrcrgllenelklmeefvkqyksealgvgev
alpgqgglpkeegkqqekpegaettaattngslsdpsKeveyvcelckgaappdspvvysdragynkqwhptcfvcakcseplvdliyfwkdgapw
cgrhyceslrprcsgcdeiifaedyqrvedlawhrkhfvcegceqllsgrayivtkgqllcptcskskrs

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Identification of LMCD1 Markers for Cancer Metastasis

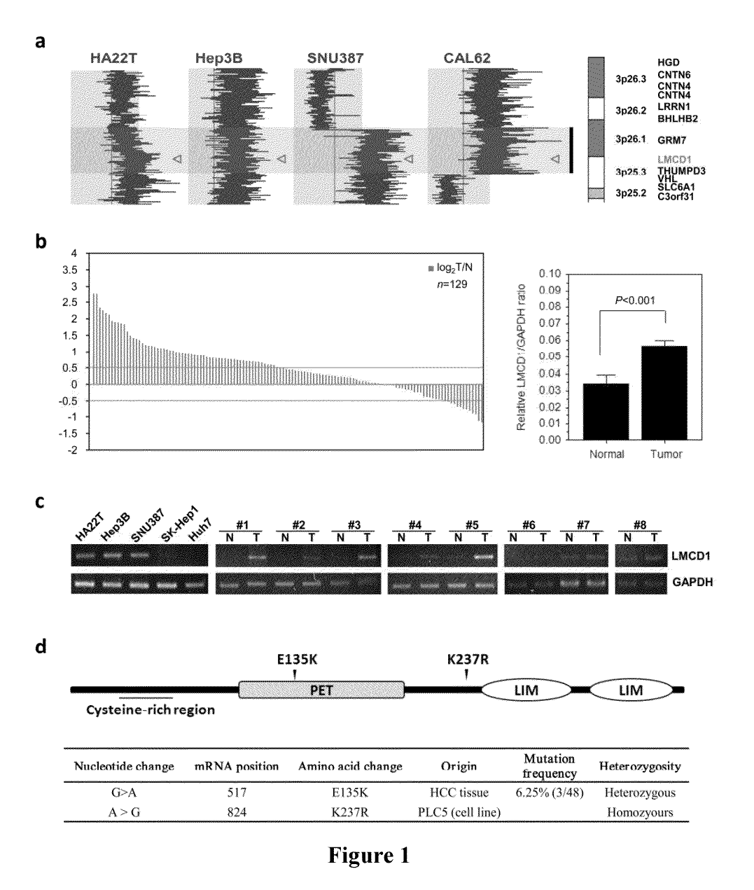

To search for putative cancer related genes in a high resolution genomic approach, a genome-wide DNA copy number alteration study in multiple human cancer cell lines was performed using Affymetrix 500K SNP GeneChip Arrays and non-matched normal reference genomes as controls. Median smoothing method was used with a window size of 5 continuous SNPs to minimize the data variation and obtain inferred copy number (ICN) of each SNP. An overlapped amplified region on chromosome 3p was detected in three HCC cell lines: HA22T (ICN=2.71), Hep3B (ICN=2.76) and SNU387 (ICN=3.32). By incorporating a 3p amplified region from thyroid carcinoma CAL62 cell line (ICN=3.46), the candidate region was narrowed to a 2.53 Mb amplicon at 3p26.1-25.3 (FIG. 1a). Among 8 known and 6 predicted genes residing in this region, LMCD1 was chosen as a candidate cancer gene based on the following three reasons: (1) LMCD1 is located at the amplification peak of overlapped amplicons in SNU387 and Hep3B cells; (2) several LIM domain containing proteins have been shown to be relevant to tumor progression (Bagheri-Yarmand et al., Int. J. Cancer. 2006 (118):2703-10; Jagadeeswaran et al., Cancer Res. 2008 (68):132-142).; and (3) LMCD1 is commonly over-expressed in HCC tissues based on in silico analysis of HCC datasets from iCOD database (61 of 129, log₂T/N>0.5), with a significantly higher average LMCD1/GAPDH level in HCCs than in normal references (P<0.001 by two-sample t test) (FIG. 1b). Based on comprising LIM and central PET domains, LMCD1 is classified into Testin subfamily. The up-regulated LMCD1 was further validated in these three HCC cell lines HA22T, Hep3B, SNU387 and 8 pairs of HCC tissues using semi-quantitative RT-PCR assay (FIG. 1c).

All of the six exons of LMCD1 were sequenced in genomic DNA samples from 48 HCC patients and 13 human HCC cell lines for screening of somatic mutation. To confirm the nucleotide alterations, forward and reverse sequencing reactions were performed. Two mutations were further confirmed by sequencing of cDNAs converted from mRNA samples to ensure expression of mutant LMCD1 in HCC tissues and cell line. Sequencing analysis of all exons of LMCD1 revealed that a recurrent point mutation, G517A (E135K) in the PET domain, occurred in 3 of 48 HCC cases (6.25%). In PLC/PRF/5 HCC cell lines, another A824G (K237R) mutation just before the first LIM domain was also identified (FIG. 1d). The expression of mutant alleles were further validated by DNA sequencing of cDNAs converted from mRNAs of HCC tissues and PLC/PRF/5 cell lines. These results indicate that LMCD1 is a possible hotspot of genetic alterations in HCC.

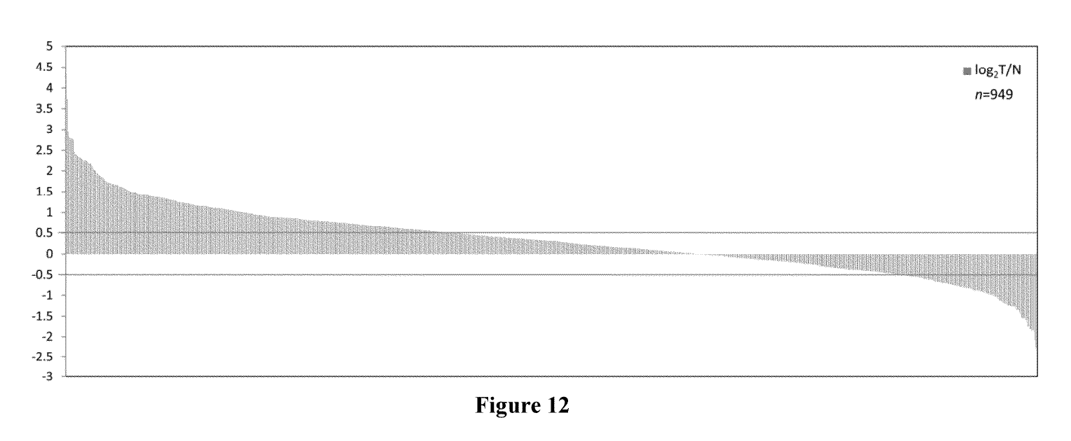

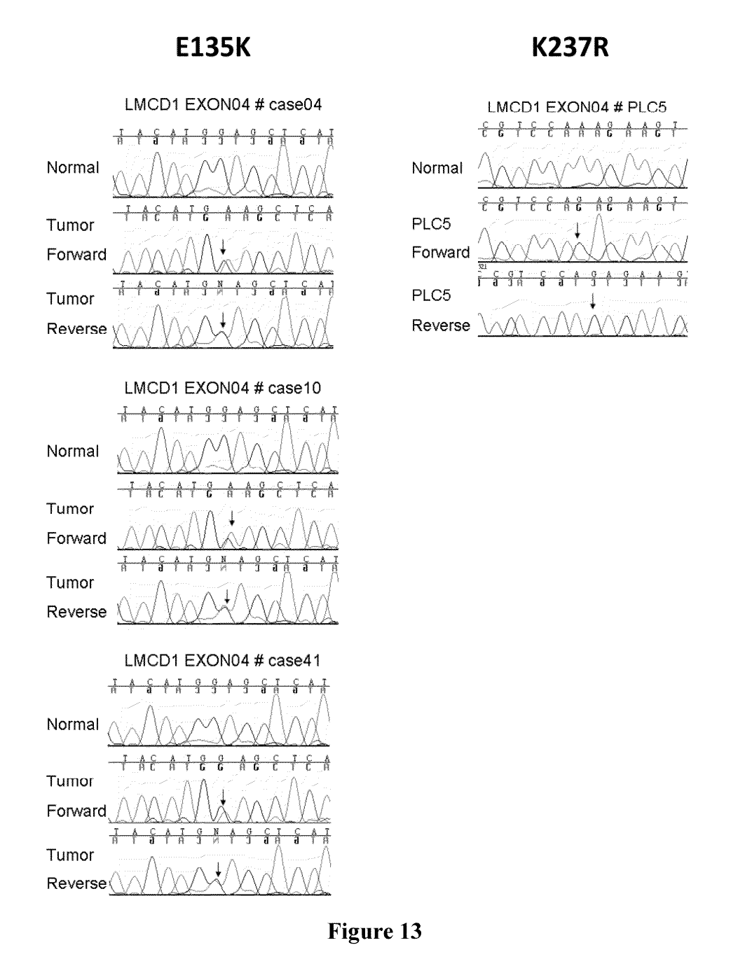

To further validate LMCD1 up-regulation in HCC tissues, additional analyses of LMCD1 expression was performed in 14 downloaded HCC datasets from public domains and revealed 377 LMCD1 over-expressing cases from 949 HCC patients (one-sample t test, P<0.001) (FIG. 12). The samples were obtained from the public repository Gene Expression omnibus (GEO) and Integrated Clinical Omics Database (iCOD). Samples of human primary liver cancer tissues were selected. For samples from two-color microarray platforms, the ratios were retrieved directly. For samples from one-color microarray platforms, the values of tumor samples were divided by the values of pooled normal samples to get tumor/normal ratios. Logarithms of base 2 were then applied to all ratios. When LMCD1 up-regulation was analyzed using iCOD HCC datasets with some clinicopathological features, LMCD1 up-regulation (log₂T/N over 0.5) was observed in 48 of the 107 patients (44.86%) with expansive tumor growth pattern and 12 of 16 (75%) with infiltrative growth pattern (P=0.0319, Fisher's Exact Test). The positive correlation of up-regulated LMCD1 with infiltrative tumor growth pattern, which is frequently observed in HCC specimens with intrahepatic metastasis, suggests possible involvement in tumor cell invasiveness. Furthermore, G517A (E135K) and A824G (K237R) were detected in tumor tissues adjacent to normal tissue lacking these mutations in the same subject (FIG. 13). LMCD1 mutations also occurred in nasopharyngeal carcinoma patients. A summary of genotyping and sequencing data is provided in Table 1.

TABLE 1

| | Location | marker | Gene | Mutation information |
|---|---|---|---|---|
| NPC | 3p25.3 | D3S4545 | LMCD1 | Tissue: E135K (2/48) |
| | | | | R154C (1/48) |
| | | | | G149G (2/48) |
| HCC | 3p25.3 | D3S4545 | LMCD1 | Tissue: E135K (3/48) |
| | | | | G149G (4/48) |
| | | | | Cell line—PLC5: K237R, G149G |
| | | | | Hep3B: G149G |

### Example 2

### LMCD1 Mutations Induce Lamellipodia Formation

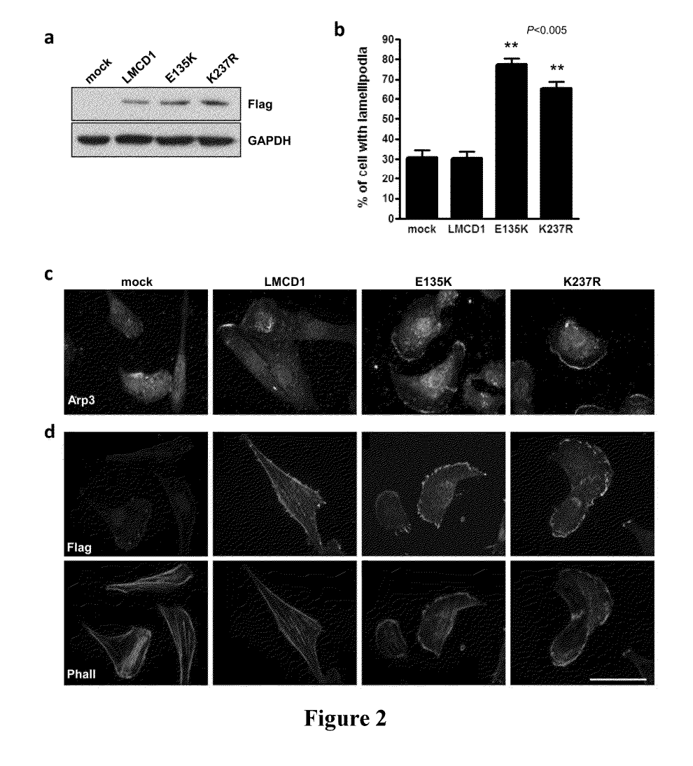

An LMCD1 expressing plasmid was constructed by inserting the PCR-amplified LMCD1 full-length fragment into pcDNA3.0-Flag vector. E135K and K237R point mutations were introduced by PCR-based mutagenesis using primer pairs 5′-CAGTA CATGAAGCTCATCCCC-3′ and 5′-GGGGATGAGCTTCATGTACTG-3′ for E135K, 5′-GACCCGTCCAGAGAAGTGGAA-3′ and 5′-TTCCACTTCTCTGGAC GGGTC-3′ for K237R. The sequences of LMCD1 mutations were verified using DNA sequencing. Plasmids encoding the wild-type (wtLMCD1) and mutant (LMCD1-E135K and LMCD1-K237R) were used to establish SK-Hep1 transfectants stably expressing the respective encoded LMCD1 proteins, and expression was confirmed by Western blot analysis (FIG. 2a). In general, except for human HCC cell line SNU387 maintained in RPMI 1640 medium, rat liver epithelial cell WB, human HEK293T cells and other human HCC cell lines including Hep3B, HA22T, SK-Hep1, Huh7 and PLC/PRF/5 were cultured in Dulbecco's modified Eagle's medium (DMEM) containing 10% fetal bovine serum, 1% nonessential amino acids, 1% penicillin-streptomycin and incubated at 37° C. under a humidified atmosphere with 5% CO₂. To establish stable cell lines, cells were seeded at 80% confluence one day before transfection. Transfection with expression constructs or corresponding empty vector (mock) was carried out by lipofectamine 2000 (Invitrogen) according to the manufacturer's protocol. Stable transfectants were obtained through selection of G418-resistant clones.

In contrast to most wtLMCD1 cells displaying classical spindle-shaped epithelial morphology similar to that of mock cells, a significant high proportion of cells expressing LMCD1-E135K and LMCD1-K237R protruded apparent lamellar extensions (77.43% and 65.56% of E135K and K237R cells, respectively, compared with 28.71% of mock cells and 31.24% of wtLMCD1 cells) (FIGS. 2b and 2c). By staining with lamellipodia marker Arp3, the significant enrichment of Arp3 staining indicated the increased formation of lamellipodia in the leading edge of LMCD1-E135K and LMCD1-K237R cells (FIG. 2c).

Subcellular localization of LMCD1 was examined by immunofluorescence staining with anti-Flag antibody, together with rhodamine-phalloidin for F-actin visualization. Although wtLMCD1 was widely expressed in the nucleus and cytoplasm, a portion of cytoplasmic wtLMCD1 was shown to co-localize with stress fibers (FIG. 2d). LMCD1-E135K and LMCD1-K237R expression somewhat reduced the stress fiber assembly but predominantly augmented and co-localized cortical actin accumulation, which was consistent with their capability to induce lamellipodial extension. These results indicate that LMCD1 may play a role in the regulation of actin cytoskeleton organization to modulate cell morphology and mutants LMCD1 redistributes the F-actin pool from stress fiber to lamellipodia.

### Example 3

### LMCD1 Mutations Promote Cell Migration

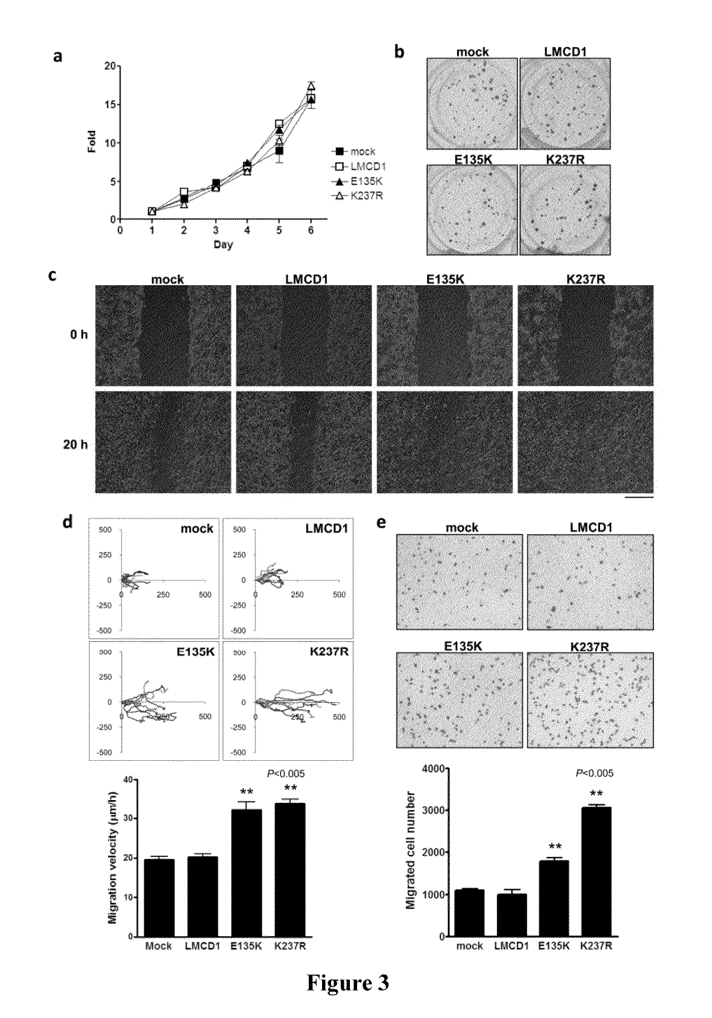

To evaluate the functions of wild type and mutant LMCD1 in HCC tumorigenesis, SK-Hep1 transfectants were examined for cell proliferation by Alamar Blue assay (AbD Serotec, UK) and transformation by anchorage-independent growth assay in soft agar. The fluorescence in the Alamar Blue assay was measured with excitation and emission wave lengths of 560 nm and 590 nm, respectively. For anchorage independent growth assay, cells were dispersed in 0.3% top agarose and plated onto 0.6% bottom agarose in culture medium. Colonies were allowed to grow for 21 days and visualized by staining with 0.05% crystal violet (Sigma-Aldrich) and photographed. All experiments were performed in triplicates. Cell proliferation and in vitro cellular transformation in mock control and all transfectants were highly similar (FIGS. 3a and 3b).

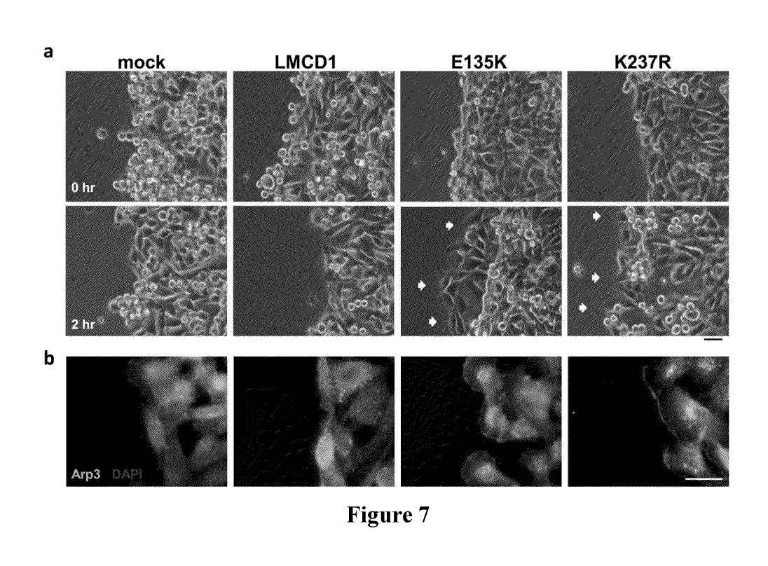

To test the hypothesis that LMCD1 mutants play a role in cell migration regulation, three different cell migration assays for reflecting alteration of directed migration, random migration, and chemotaxis were performed. Directed migration of these SK-Hep1 transfectants was inspected by wound healing assay. For the wound healing assay, cells were seeded in a 6-well plate at a confluent density and cultured overnight to grow into a monolayer. The culture was scratched with a p200 pipette tip, and the wound was allowed to close for 20 hours. The average reduction of distance between two wound edges was calculated to evaluate the rate of wound closure. Lamellipodia formation of LMCD1-E135K and LMCD1-K237R cells at the wound edge was conspicuous and could be detected as early as two hours after wounding (FIG. 7). Cells were photographed immediately after wounding (FIG. 7a upper) and 2 hours after wounding (FIG. 7a lower), with arrows indicating protrusion of cells at the wound edge. Two hours after wounding, cells were fixed with 4% paraformaldehyde in PBS for 10 minutes, permeabilized with 0.1% Triton X-100 in PBS for 10 minutes, blocked with 3% BSA in PBS for one hour at room temperature, and then stained with anti-Arp3 antibody for lamellipodia and DAPI for the nucleus (FIG. 7b; bar=50 μm). Primary antibodies and the corresponding Alexa Fluor 488-conjugated secondary antibodies were applied for one hour each. Rhodamine-phalloidin for F-actin staining was incubated with cells for 10 minutes. Coverslips were mounted on slides using VECTASHIELD mounting medium with DAPI (Vector Laboratories). Images were obtained using an Olympus BX51 fluorescence microscope or a PerkinElmer Ultra View confocal microscope (PerkinElmer Life Sciences). Accelerated wound closure was observed for LMCD1-E135K and LMCD1-K237R cells (FIG. 3c).

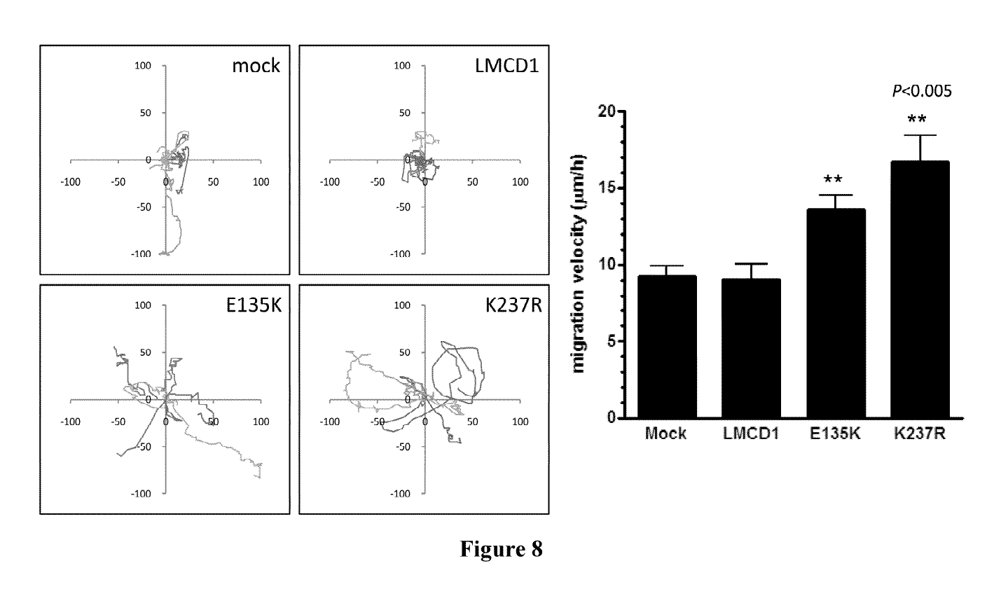

To further monitor the morphology of wound-edge migrating cells, live-cell imaging was performed. The LMCD1-E135K and LMCD1-K237R cells moved significantly faster than mock or wtLMCD1 cells with more dynamically prominent lamellipodia formation. Tracking of randomly selected cells revealed advanced directed migration velocity of 1.65 and 1.75 fold for LMCD1-E135K and LMCD1-K237R cells, respectively, compared with mock or wtLMCD1 cells (FIG. 3d). Traces in FIG. 3d represent the tracked movement of individual cells during wound closure, transposed onto the axis with the origin as the starting point for each cell. For tracking movement, cells were seeded into a 60 mm dish and placed in a CO₂— and temperature-controlled chamber on an Axiovert 200M microscope (Carl Zeizz MicroImaging, Inc.). Cell migration was recorded under a 20× objective lens with a CCD video camera (CollSNAP fx; Roper Scientific). Time-lapse images were captured at 15-minute intervals over 18 hours. Average cell velocities are quantified in the bar graph below the traces (±SEM). Similar results were observed in random migration assay, in which average migration velocity for LMCD1-E135K and LMCD1-K237R cells was increased to about 1.47 and 1.8 fold, respectively, compared to mock or wtLMCD1 cells (FIG. 8). The traces in FIG. 8 were obtained as for FIG. 3d, by manually tracking the movement of individual cells during an 18-hour time-lapse interval, with images taken every 15 minutes, and positions transposed onto an axis with the origin as the cell's starting point. Bar graphs in FIG. 8 represent quantification of migration velocity means (±SEM).

For trans-well assay, 1×10⁴ cells resuspended in serum-free medium were seeded into the upper chamber of an insert with an 8.0-μm pore size of polycarbonate membrane (BD Biosciences) and then placed into the bottom chamber containing 10% FBS as chemoattractant. Cells were allowed to migrate for 24 hours followed by methanol fixation and Giemsa staining (MERCK Darmstadt, Germany). Un-migrated cells on the membrane apical side were removed. Migrated cells were photographed with a phase contrast microscope and the number was counted by using Image-Pro Plus software. All experiments were performed in triplicates. Consistent with the other migration experiments, expression of LMCD1-E135K and LMCD1-K237R resulted in 1.62 and 2.78 fold increase in cell chemotaxis mobility in trans-well migration assay (FIG. 3e). Images in the top panel of FIG. 3e are representative of three independent experiments performed in triplicate. Quantification of average migrated cell number is shown in bar graphs below the images (±SEM).

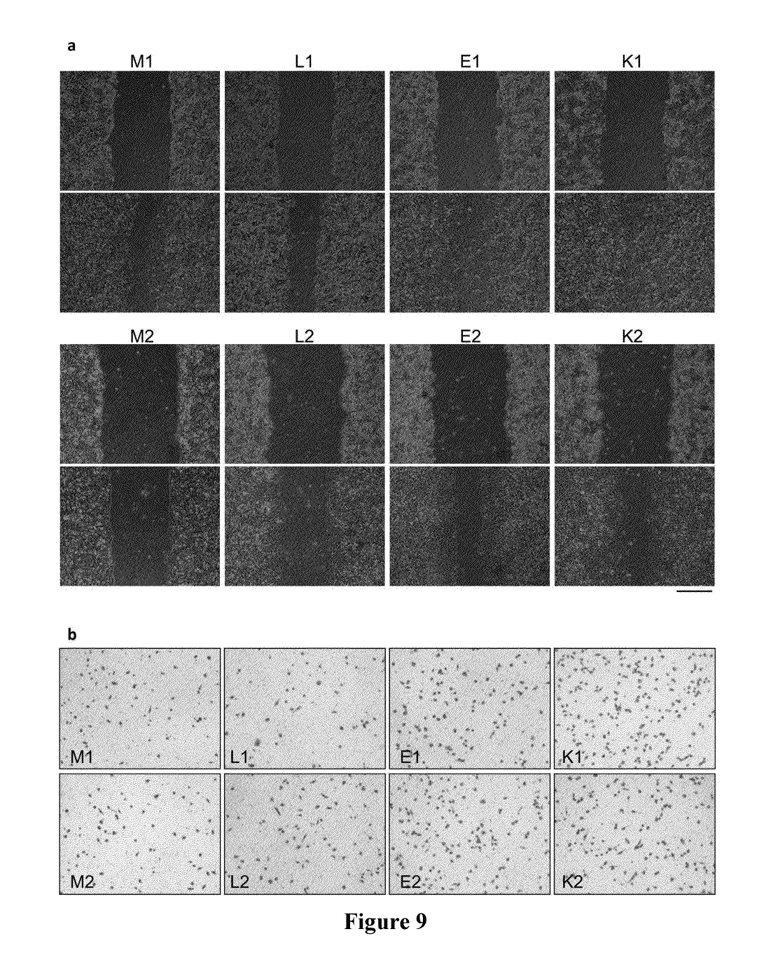

To avoid selection bias of transfected clones to enhance cell migration, another set of independent clones were examined with the same migration experiments and obtained similar results (FIG. 9). FIG. 9a shows the results of wound healing assays with two independent LMCD1 stable transfectants in SK-Hep1 cells. FIG. 9b provides representative images for the trans-well assay for these two independent LMCD1 clones. In FIG. 9, the labels M1 and M2 represent two transfectants of mock; L1 and L2 represent wild-type LMCD1 (wtLMCD1); E1 and E2 represent LMCD1-E135K transfectants; and K1 and K2 represent LMCD1-K237R transfectants.

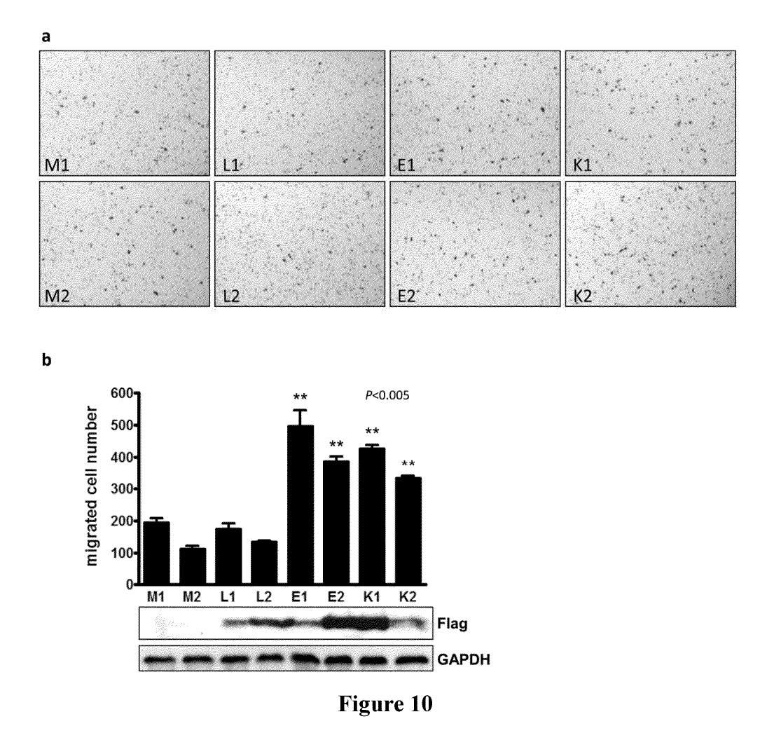

In addition, LMCD1-expressing Huh7 cells were also established to clarify whether the altered functions of E135K and K237R mutations are similar in other HCC cell lines. Two distinct clones of Huh7 transfectants for each construct with different protein expression levels were selected to test in the trans-well migration assay. Similar to the results in SK-Hep1 cells, expression of LMCD1-E135K and LMCD1-K237R but not that of wtLMCD1 in Huh7 transfectants increased cell migration (FIG. 10). The LMCD1 transfectants in Huh7 cells (5×10⁴ cells) were examined for trans-well migration for 24 hours, with images representative of three independent experiments performed in triplicate shown in FIG. 10a. Quantification of average migrated cell number is shown in bar graphs of FIG. 10b (±SEM), which also illustrates LMCD1 protein expression as determined by Western blot.

Taken together, these results indicate that LMCD1-E135K and LMCD1-K237R do not significantly alter cell proliferation and transformation capabilities but do enhance cell migration in multiple HCC cells.

### Example 4

### Suppression of LMCD1 by RNAi Reverses Migration Effects

Five independent pLKO.1 lentiviral-based shRNA clones targeting human LMCD1 (NM_{—}014583) from The RNAi Consortium (TRC) library were tested. Only one construct (number TRC shRNA, TRCN0000134556 5′-CCGGGAATGAGTTGAAACTGATGGACTCGAGTCCATCAGTTTCAACTCATTCTTTTTTG-3′) displayed significant knockdown efficiency and was used in further analyses. Human shRNA for RAC1 (shRac1) was also obtained from TRC library with number TRCN0000004870: 5′-CCGGGCTAAGGAGATTGGTGCTGTACTCGAGTACAGCACCAATCTCCTTAGCTTTTT-3′. pLKO.1-shLuc was used as control. To generate shRNA lentiviral particles, HEK293T cells were co-transfected with a plasmid mixture (pLKO.1-puro-shRNA, pCMVΔR8.91 packaging plasmid, and pMD.G envelope plasmid) by calcium phosphate protocol. Cell medium containing viral particles was collected three times at 24-hour intervals after transfection. The virus-containing medium was aliquoted and used to infect cells or stored at −80° C. until use. Ninety-six hours after infection, cells were subjected to further experimental assays or harvested for LMCD1 or Rac1 gene expression analysis by RT-PCR or Western blotting.

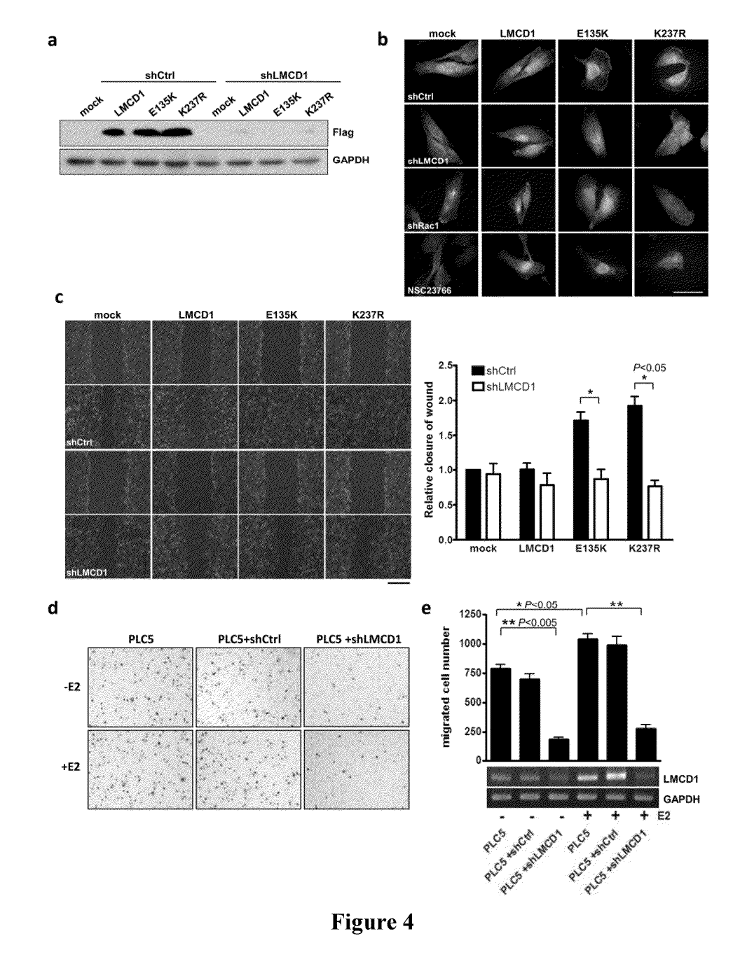

In order to validate the LMCD1-E135K and LMCD1-K237R enhanced cell migration, LMCD1 expression was knocked down by shLMCD1 lentiviral infection. FIG. 4a shows the results of a Western blot analysis of LMCD1 knockdown efficiency in multiple stable clones infected with lentiviral shLMCD1 or luciferase shRNA (shCtrl) as control, confirming specific knockdown of LMCD1 at the protein level. LMCD1 knockdown did not change the cell morphology of mock and wtLMCD1 cells. However, the enriched Arp3 in lamellipodia and periphery extension of LMCD1-E135K and LMCD1-K237R cells were no longer observed after introduction of shLMCD1 (FIG. 4b). Columns in FIG. 4b represent the cell treated (mock transfected, wtLMCD1 transfected, or mutant LMCD1 transfected), with indicated shRNA or inhibitor (NSC23766, Rac1 inhibitor) treatment indicated by row (bar=50 μm). In agreeing with their decreased lamellipodia, shLMCD1 also diminished wound closure rate of LMCD1-E135K and LMCD1-K237R cells to a level similar to that of mock or wtLMCD1 cells (FIG. 4c). Wound closure assays were conducted in triplicate, as described in Example 3. Cells treated are indicated in column headers, and treatment is indicated in the rows (bar=50 μm). The bar graph to the right of the images in FIG. 4c provides quantification of average migration results, normalized to the mock cells (±SEM). These results confirm that induced lamellipodia formation and cell migration indeed arise from mutant LMCD1 expression.

To demonstrate whether endogenous LMCD1-K237R mutation modulates cell mobility in PLC/PRF/5 cells, we induced LMCD1 expression by estrogen treatment as reported in breast cancer cells (see e.g. Dudek and Picard, PLoS One, 3: 674-687 (2008); and Harvell et al., Endocrinology, 147:700-713 (2006)) and examined the migration alteration of PLC/PRF/5 cells. Trans-well migration assays were performed as in Example 3. Expression of LMCD1-K237R in PLC/PRF/5 cells (1×10⁵ cells) was induced by exposure to 10 nM 17β-estradiol. Results are provided in FIG. 4d, with some cells also being treated with the indicated shRNA. Images in FIG. 4d are representative of migration results from three independent experiments, with (+E2) and without (−E2) estradiol treatment. The migration results are quantified by bar graph in FIG. 4e (±SEM). Effects estradiol and shRNA treatment on the expression of LMCD1-K237R were confirmed by reverse-transcription PCR using LMCD1-specific primer pairs, and GAPDH as an internal control (FIG. 4e, bottom). Our results demonstrated that addition of estrogen to PLC/PRF/5 could indeed up-regulate expression of LMCD1-K237R and cell migration. Further treatment with shLMCD1 abrogated endogenous LMCD1-K237R expression and impaired cell mobility concomitantly with or without estrogen induction (FIGS. 4d and 4e). These data provide evidence that LMCD1-K237R mutation at an endogenous level is necessary for PLC/PRF/5 cell mobility regardless the estrogen induction.

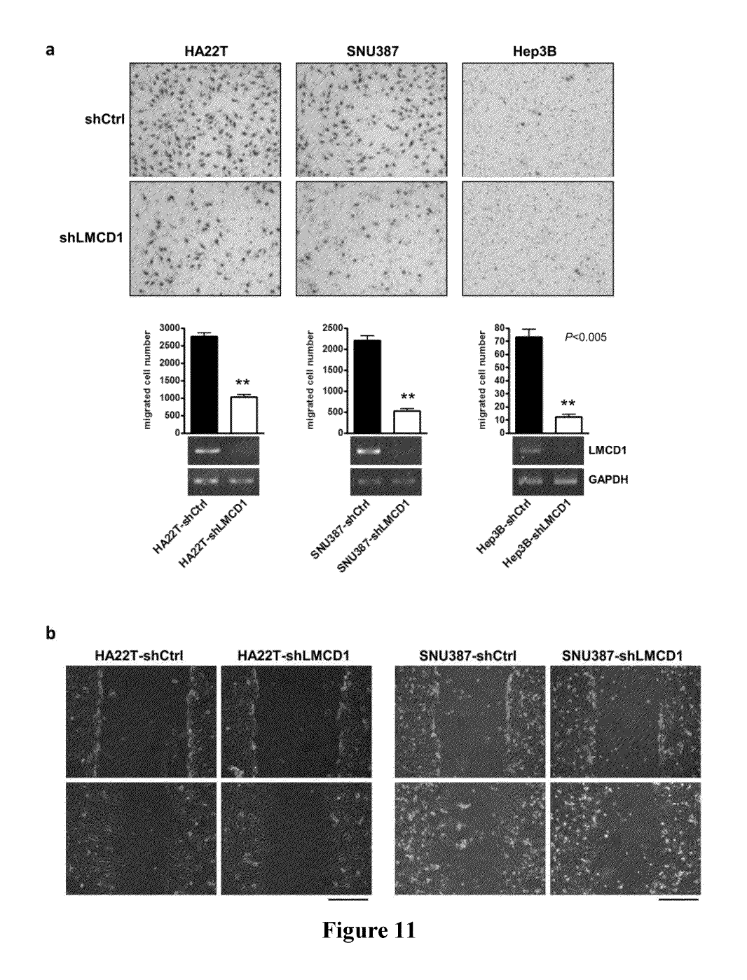

shLMCD1 knockdown assay was also performed in HCC cells HA22T, SNU387 and Hep3B carrying LMCD1 amplicon to examine the role of up-regulated wtLMCD1 in cell migration. The decrease of cell migration in trans-well and wound healing assays indicate that up-regulation of LMCD1 thru amplification is a significant factor for promoting cell migration in HCC cells (FIG. 11). FIG. 11a shows that shLMCD1 treatment in the indicated cells significantly decreased cell migration by trans-well migration assay and significantly diminished expression of LMCD1 by semi-quantitative RT-PCR. FIG. 11b shows that shLMCD1 treatment in the indicated cells significantly reduced wound closure (bar=500 μm).

### Example 5

### Rac1 Involved in Mutant LMCD1-Mediated Migration Increase

To test the possibility that Rac1, a key regulator of lamellipodia formation, is involved in enhanced lemellipodia formation and mobility induced by LMCD1 mutation, shRNA and a specific inhibitor of Rac1, shRac1 and NSC23766, respectively, were applied to LMCD1 transfectants. Results described in Example 4 indicated that treatment of shRac1 or NSC23766 inhibited lamellipodia extension of LMCD1-E135K and LMCD1-K237R cells (FIG. 4b).

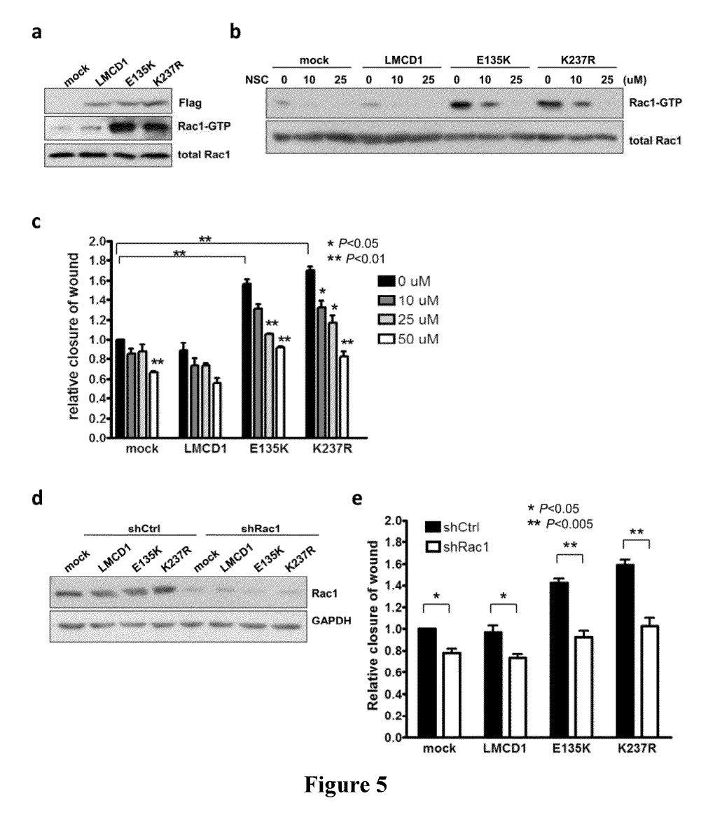

Rac1 activity was also assayed during wound healing migration. Rac1 activity measurement was performed by affinity pull-down assay using a glutathione S-transferase (GST) fusion protein conjugated with the p21 binding domain derived from a Rac1 effector, PAK1 (GST-PAK1 PBD). Cells were lysed in lysis buffer containing 25 mM HEPES, pH 7.5, 150 mM NaCl, 1% Nonidet P-40, 10 mM MgCl₂, 1 mM EDTA, 10% glycerol, 1 mM PMSF, 1× protease inhibitor cocktail (Roche Diagnostics), 1 mM sodium vanadate, and 10 mM NaF. Cell lysates were normalized by Bradford protein assay for equal amount of protein and incubated with GST-PAK1 PBD coupled glutathione-Sepharose 4B beads for 1 h at 4° C. The beads were washed with lysis buffer five times then boiled in SDS-PAGE loading buffer. Samples were analyzed by SDS-PAGE and Western blot to detect the level of Rac1-GTP with anti-Rac1 antibody. Included was a Western blot analysis of total and GTP-bound Rac1 expression in LMCD1 transfectants after repeated scratching of cells. LMCD1-E135K and LMCD1-K237R cells exhibited increased Rac1 activation level whereas wtLMCD1 over-expression did not influence endogenous Rac1 activity (FIG. 5a; cells indicated by column labels, detected protein indicated by row label).

To further confirm that mutant LMCD1-mediated cell migration enhancement was Rac1 dependent, we performed Western analysis and wound healing assays in the presence of NSC23766. To determine the inhibitory effect of Rac1 inhibitor on wound closure, cells were pre-treated with indicated amount of NSC23766 for 6 hours. The same drug concentration was also contained in each medium during the migration period. The addition of NSC23766 decreased the Rac1 activity and the cell mobility in a dose dependent manner (FIGS. 5b and 5c). Cells and dose of inhibitor are indicated by column in FIG. 5b, with detected protein indicated by row. The data in FIG. 5c represent means±SEM. Treatment with 25 μM NSC23766 caused 31˜32.3% migration inhibition for LMCD1-E135K and LMCD1-K237R mutant transfectants in compared with only 12.1˜16.4% inhibition in mock and wtLMCD1 cells (FIG. 5c). Treatments with shRac1 had similar inhibitory effect on mutant LMCD1-mediated cell migration (FIGS. 5d and 5e). Cells and shRNA are indicated by column in FIG. 5d, with detected protein indicated by row. Results in FIG. 5e represent means of degree of wound closure, normalized to that of mock cells, ±SEM. These results indicate that LMCD1-E135K and LMCD1-K237R transfectants addicted to the mutation-mediated increase of cell migration and are more sensitive than wtLMCD1 transfectant to Rac1 inhibitor or shRNA.

### Example 6

### Promotion of Metastasis by LMCD1 Mutation

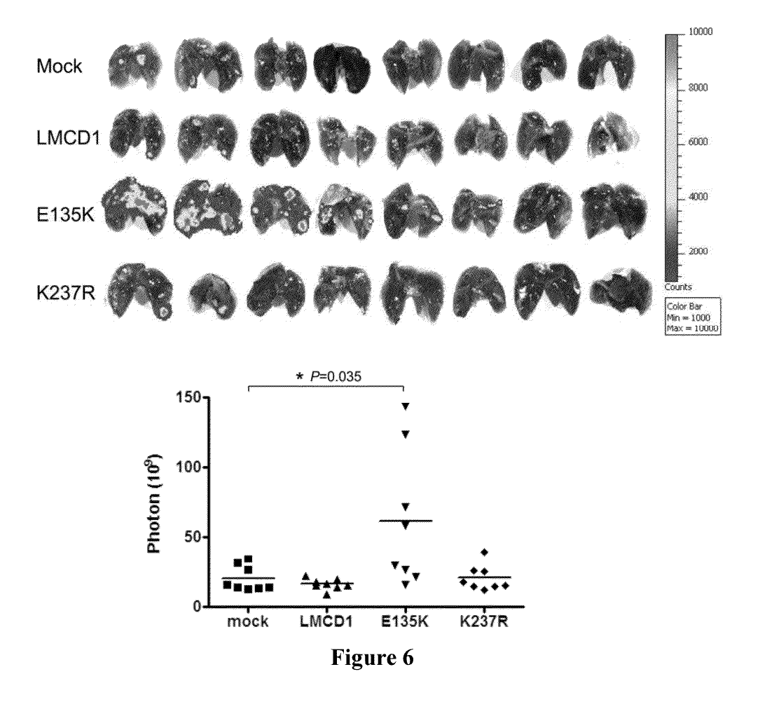

To further investigate the influence of LMCD1 mutations on tumor metastasis in vivo, RFP-labeled LMCD1 transfectants of SK-Hep1 cells were established for tail vein injection in a nude mouse metastasis model. LMCD1 transfectants were infected with RFP lentivirus and sorted for RFP-positive cells. Expanded cells (1×10⁶ per mouse) were i.v. injected into athymic BALB/c nude (nu/nu) mice at 4 weeks age. Mice were sacrificed 8 weeks after injection. Lung tissues were isolated and RFP-labeled metastatic cells were quantified using photon counting technique of Living Image software. Lung metastatic signals were detected using IVIS system (Xenogen Corp., Alameda, Calif.) with excitation and emission wave length at 570 and 620 nm, respectively. Photons emitted from lung metastases were quantified by Living Image software (Xenogen Corp., Alameda, Calif.). All animal works were performed in accordance with the guidelines of the Institutional Animal Care and approved by the Animal Committee of Academia Sinica. Our results showed that mock and wtLMCD1 transfectant displayed weak intrinsic metastatic ability while LMCD1-E135K transfectant (mutations identified in HCC tissues) significantly promoted lung metastasis (FIG. 6). FIG. 6 provides both images of the lungs analyzed, as well quantification of photon counts (with higher indicating greater metastasis) in the graph below the images. There was no significant increase in tumor metastases formed by LMCD1-K237R (mutation identified in PLC/PRF/5 cell) transfectant in this model system. Nevertheless, the capability of LMCD1-E135K to promote systemic metastasis and its clinical occurrence in HCC patients make LMCD1-E135K importance with regard to HCC progression.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for characterizing a cancer tissue comprising:
(a) providing a sample of a subject;
(b) assaying said sample for the presence or absence of one or more LMCD1 mutations by detecting the presence or absence of the one or more LMCD1 mutations or a SNP in high linkage disequilibrium with said one or more LMCD1 mutations, wherein the presence of said one or more LMCD1 mutations indicates a higher propensity for metastasis than is indicated by the absence of said one or more LMCD1 mutations; and
(c) reporting the result of step (b) to a designated person or entity.

2. The method of claim 1, wherein said one or more LMCD1 mutations is one or more of G517A (SEQ ID NO: 18) and A824G (SEQ ID NO: 19).

3. The method of claim 1, wherein said one or more LMCD1 mutations is one or more of E135K (SEQ ID NO: 20) and K237R (SEQ ID NO: 21).

4. The method of claim 1, wherein said cancer tissue comprises hepatocellular carcinoma or nasopharyngeal carcinoma.

5. The method of claim 1, wherein said assaying comprises contacting a nucleic acid derived from said sample with an oligonucleotide in a hybridization reaction.

6-7. (canceled)

8. The method of claim 1, wherein said assaying comprises contacting a protein derived from said sample with a binding element specific for an LMCD1 mutant protein, wherein detection of binding between said binding element and said LMCD1 mutant protein indicates the presence of one of said one or more LMCD1 mutations.

9. (canceled)

10. The method of claim 1, further comprising the step of administering a therapeutic agent based on the results of step (b).

11. The method of claim 10, wherein said therapeutic agent is an inhibitor that downregulates Rac1 output.

12. The method of claim 11, wherein said downregulation of Rac1 output is indicated by one or more of:
(a) a decreased number of migrated cells in a trans-well migration assay;
(b) a decrease in migration velocity of cells in a wound-healing assay;
(c) a decrease in migration velocity of cells in a random migration assay;
(d) a decrease in the percentage of cells with lamellipodia; and
(e) a decrease in Rac1 expression at the nucleic acid or protein level;
wherein each of (a)-(e) compares treated and untreated cells expressing LMCD1 comprising said one or more LMCD1 mutations.

13. The method of claim 1, wherein a higher propensity for metastasis induced by said one or more LMCD1 mutations is indicated by one or more of:
(a) an increased number of migrated cells in a trans-well migration assay;
(b) an increase in migration velocity of cells in a wound-healing assay;
(c) an increase in migration velocity of cells in a random migration assay; and
(d) an increase in the percentage of cells with lamellipodia;
wherein each of (a)-(d) compares cells expressing LMCD1 comprising said one or more LMCD1 mutations and cells expressing LMCD1 lacking said one or more LMCD1 mutations.

14. A method of predicting propensity of cancer cells to metastasize, the method comprising:
(a) providing a sample of a subject;
(b) assaying said sample for the presence or absence of one or more LMCD1 mutations; and
(c) predicting the propensity of said cancer cells to metastasize based on the presence or absence of the one or more LMCD1 mutations.

15. The method of claim 14, wherein the presence of said one or more LMCD1 mutations indicates a higher propensity for metastasis than is indicated by the absence of said one or more LMCD1 mutations.

16-27. (canceled)

28. The method of claim 1, further comprising step (d) treating said subject with an inhibitor that reduces metastasis, if said one or more LMCD1 mutations is present.

29-42. (canceled)

43. A method for screening inhibitors of cancer metastasis comprising:
(a) providing a cell line expressing LMCD1 having one or more LMCD1 mutations;
(b) exposing said cell line to a test compound; and
(c) determining the effect of said compound on cell migration, wherein a decrease in cell migration of cells treated with said compound compared to cells not treated with said compound identifies said compound as an inhibitor of cancer metastasis.

44-52. (canceled)

53. An isolated oligonucleotide for the detection of an LMCD1 mutation according to the method of claim 1, wherein said isolated oligonucleotide comprises at least 6 nucleotides complementary to a target sequence comprising said LMCD1 mutation, wherein said at least 6 nucleotides comprise at least one nucleotide complementary to said LMCD1 mutation, and further wherein said isolated oligonucleotide is substantially complementary to said target sequence.

54-57. (canceled)

58. An isolated antibody, or antigen-binding antibody fragment thereof, for the detection of an LMCD1 mutation according to the method of claim 1, wherein said antibody or antigen-binding antibody fragment is directed specifically to a human LMCD1 mutant protein, or protein fragment thereof comprising one or more LMCD1 mutations.

59-65. (canceled)

66. An expression vector for use in the method of claim 43, wherein said expression vector comprises a nucleotide sequence encoding a mutant LMCD1 comprising one or more LMCD1 mutations, wherein expression of said mutant LMCD1 increases cell mobility in cells that actively express said expression vector.

67-71. (canceled)

72. A transgenic cell for use in the method of claim 43, wherein the genome of said transgenic cell comprises a stably integrated transgenic nucleotide sequence encoding a mutant LMCD1 comprising one or more LMCD1 mutations, wherein said transgenic cell actively expresses said mutant LMCD1 and exhibits increased cell mobility relative to a cell not expressing said mutant LMCD1.

73-77. (canceled)

78. A non-human transgenic animal for use in the method of claim 43, wherein the genome of said non-human transgenic animal comprises a stably integrated nucleotide sequence encoding a mutant LMCD1 comprising one or more LMCD1 mutations, wherein cancer cells arising from the cells of said non-human transgenic animal and expressing said mutant LMCD1 exhibit a higher propensity for metastasis relative to cancer cells not expressing said mutant LMCD1.

79-84. (canceled)

85. A kit for assaying for a mutation of LMCD1 according to the method of claim 1, wherein the kit comprises:
(a) one or more reagents suitable for detecting a mutant LMCD1 comprising one or more LMCD1 mutations; and
(b) instructions for the use of said one or more reagents.

86-87. (canceled)
